(19) United States
(12) Patent Application Publication
(10) Pub. No.: US 2011/0229933 A1
(43) Pub. Date: Sep. 22, 2011

(54) HIGH TITER ANTIBODY PRODUCTION

(75) Inventors: Rajesh Krishnan, Annadale, NJ (US); Dennis Rendeiro, Cedar Grove, NJ (US); Sandeepa Sandadi, Iselin, NJ (US)

(73) Assignee: SAchering Corporaton, Kenilworth, NJ (US)

(21) Appl. No.: 13/120,558

(22) PCT Filed: Sep. 24, 2009
(86) PCT No.: PCT/US2009/058164

§ 371 Date: Jun. 8, 2011

Related U.S. Application Data
(60) Provisional application No. 61/100,450, filed on Sep. 26, 2008.

(51) Int. Cl. C12P 21/00; C12N 5/07; C12M 3/00

(52) U.S. Cl. 435/69.6; 435/69.1; 435/404; 435/289.1; 435/296.1

(57) ABSTRACT

The present invention provides, in part, methods for recombinantly expressing proteins at a high level along with cell culture media for doing the same.

This application claims the benefit of U.S. provisional patent application No. 61/100,450; filed Sep. 26, 2008, which is herein incorporated by reference in its entirety.

### FIELD OF THE INVENTION

The present invention relates, generally, to a method for producing large quantities of a protein, such as an antibody, in a cell culture; along with the cell culture itself.

### BACKGROUND OF THE INVENTION

Culturing cells for the commercial production of therapeutic proteins is a costly process. The equipment required is expensive and research and development and production costs are high. Development of cell culture processes which maximize the quantity of therapeutic protein produced per liter of cell culture will minimize the resources necessary to produce a given quantity of the protein. It is, thus, desirable to use commercially viable processes which produce large quantities of proteins.

Many naturally occurring cells do not produce large quantities of desired proteins, under standard culture conditions. Rather, extensive research and development of cell culture processes, which coax cells in culture to generate large quantities of therapeutic protein, must be performed. Typically, identifying the optimal cell culture conditions is difficult and requires a significant amount of inventive input.

### SUMMARY OF THE INVENTION

The present invention provides, generally, methods and compositions for generating high quantities of protein from cells in culture by use of various culture supplements and other alterations to the culture conditions.

The present invention provides a method for producing a protein comprising inoculating an initial mammalian cell growth medium with host cells expressing the protein and adding supplements comprising those set forth below, optionally at the approximate indicated concentrations (not including the quantity of each component from other sources such as from the initial mammalian cell culture medium):

| | | |
|---|---|---|
| Adenine sulfate: | 1.632 | mg/liter |
| Adenosine: | 17.6 | mg/liter |
| Ammonium vanadate: | 0.00078 | mg/liter |
| Biotin: | 0.28 | mg/liter |
| Choline Chloride: | 50.2 | mg/liter |
| Cobalt chloride: | 0.0025 | mg/liter |
| Cupric sulfate: | 0.0032 | mg/liter |
| Cytidine: | 17.6 | mg/liter |
| D-Calcium Pantothenate: | 23.8 | mg/liter |
| Ethanolamine HCl: | 4.4 | mg/liter |
| Flavin Adenine Dinucleotide: | 0.05 | mg/liter |
| Folic Acid: | 4.6 | mg/liter |
| Glycine | 72 | mg/liter |
| Guanosine: | 17.6 | mg/liter |
| Hypoxanthine: | 11.8 | mg/liter |
| i-Inositol: | 73.2 | mg/liter |
| L-alanine: | 8.9 | mg/liter |
| L-arginine | 312.4 | mg/liter |
| L-asparagine: | 842 | mg/liter |
| L-aspartic acid | 97.6 | mg/liter |
| L-citrulline: | 12.6 | mg/liter |
| L-cysteine-HCl | 224 | mg/liter |
| L-cystine: | 34 | mg/liter |
| L-glutamic acid | 155.4 | mg/liter |
| L-histidine | 167 | mg/liter |
| Lipoic Acid: | 0.52 | mg/liter |
| L-isoleucine | 422 | mg/liter |
| L-leucine | 384 | mg/liter |
| L-lysine | 365 | mg/liter |
| L-methionine | 147.2 | mg/liter |
| L-ornithine-HCl: | 25.6 | mg/liter |
| L-phenylalanine | 207 | mg/liter |
| L-proline | 239 | mg/liter |
| L-serine: | 281 | mg/liter |
| L-threonine | 211.6 | mg/liter |
| L-tryptophan | 109.2 | mg/liter |
| L-tyrosine | 234 | mg/liter |
| L-valine | 308.8 | mg/liter |
| Manganese chloride tetrahydrate: | 0.0003 | mg/liter |
| Niacin: | 31.4 | mg/liter |
| Nickel dichloride hexahydrate: | 0.0004 | mg/liter |
| Progesterone: | 0.015 | mg/liter |
| Putrescine 2HCl: | 0.4 | mg/liter |
| Pyridoxine HCl: | 3 | mg/liter |
| Riboflavin: | 1.86 | mg/liter |
| Sodium molybdate dehydrate: | 0.00016 | mg/liter |
| Sodium phosphate monobasic: | 288.2 | mg/liter |
| Sodium selenite: | 0.01426 | mg/liter |
| Thiamine HCl: | 16 | mg/liter |
| Thymidine: | 7.8 | mg/liter |
| Tin chloride dehydrate: | 0.00008 | mg/liter |
| Uridine: | 17.6 | mg/liter |
| Vitamin B12: | 3.4 | mg/liter |
| Vitamin E: | 0.376 | mg/liter |
| Zinc sulfate: | 1.08 | mg/liter |
| Glucose | 1.5 | g/liter |
| L-glutamine | 150 | mg/liter |

In an embodiment of the invention, some supplements are added from an amino acid feed stock solution (e.g., a 50× stock solution) that comprises amino acids at about the following concentrations:

| | | |
|---|---|---|
| L-arginine: | 6.32 | g/liter |
| L-cystine: | 1.7 | g/liter |
| L-histidine: | 2.1 | g/liter |
| L-isoleucine: | 2.6 | g/liter |
| L-leucine: | 2.6 | g/liter |
| L-lysine: | 3.6 | g/liter |
| L-Methionine: | 0.76 | g/liter |
| L-phenylalanine: | 1.65 | g/liter |
| L-threonine: | 2.38 | g/liter |
| L-tryptophan: | 0.51 | g/liter |
| L-tyrosine: | 1.8 | g/liter |
| L-valine: | 2.34 | g/liter |

In an embodiment of the invention, about 20 ml amino acid feed stock solution is added per liter of culture. In an embodiment of the invention, the following supplements are added from an amino acid feed stock solution (e.g., a 100× stock solution) that comprises amino acids at about the following concentrations:

| | | |
|---|---|---|
| L-alanine: | 0.89 | g/liter |
| L-asparagine: | 1.5 | g/liter |
| L-aspartic acid: | 1.33 | g/liter |
| L-glutamic acid: | 1.47 | g/liter |
| Glycine: | 0.75 | g/liter |
| L-proline: | 1.15 | g/liter |
| L-serine: | 1.05 | g/liter |

In an embodiment of the invention, the amino acid feed stock solution is added at about 10 ml amino acid feed is added per liter of culture. In an embodiment of the invention, some of the supplements are added from a nutrient feed stock solution (e.g., a 50× stock solution) that comprises supplements at about the following concentrations:

| | | |
|---|---|---|
| L-asparagine: | 40.6 | g/liter |
| L-proline | 10.81 | g/liter |
| L-isoleucine | 18.53 | g/liter |
| L-cysteine-HCl | 11.19 | g/liter |
| L-leucine | 16.58 | g/liter |
| L-threonine | 8.2 | g/liter |
| L-tyrosine | 9.9 | g/liter |
| L-arginine | 9.29 | g/liter |
| L-aspartic acid | 3.56 | g/liter |
| L-glutamic acid | 6.28 | g/liter |
| Glycine | 2.83 | g/liter |
| L-histidine | 6.23 | g/liter |
| L-methionine | 6.58 | g/liter |
| L-tryptophan | 4.93 | g/liter |
| L-lysine | 14.66 | g/liter |
| L-phenylalanine | 8.64 | g/liter |
| L-valine | 13.08 | g/liter |
| L-serine: | 13 | g/liter |
| Sodium phosphate monobasic: | 14.41 | g/liter |
| Zinc sulfate: | 0.054 | g/liter |
| Cupric sulfate: | 0.00016 | g/liter |
| Ammonium vanadate: | 0.000039 | g/liter |
| Cobalt chloride: | 0.000125 | g/liter |
| Nickel dichloride hexahydrate: | 0.00002 | g/liter |
| Sodium molybdate dehydrate: | 0.000008 | g/liter |
| Tin chloride dehydrate: | 0.000004 | g/liter |
| Manganese chloride: tetrahydrate: | 0.000015 | g/liter |

In an embodiment of the invention, about 20 ml of the nutrient feed stock solution is added per liter of culture. In an embodiment of the invention, some of the supplements are added from a vitamin/salt feed stock solution (e.g., a 50× stock solution) that comprises supplements at about the following concentrations:

| Sodium selenite: | 7.13 × 10⁻⁴ | g/liter |
|---|---|---|
| Adenine sulfate: | 0.0816 | g/liter |
| Adenosine: | 0.88 | g/liter |
| Cytidine: | 0.88 | g/liter |
| Guanosine: | 0.88 | g/liter |
| Uridine: | 0.88 | g/liter |
| Hypoxanthine: | 0.59 | g/liter |
| L-citrulline: | 0.63 | g/liter |
| L-ornithine-HCl: | 1.28 | g/liter |
| Biotin: | 0.014 | g/liter |
| Flavin Adenine Dinucleotide: | 0.0025 | g/liter |
| Folic Acid: | 0.23 | g/liter |
| Lipoic Acid: | 0.026 | g/liter |
| Niacin: | 1.57 | g/liter |
| Pyridoxine HCl: | 0.15 | g/liter |
| Riboflavin: | 0.093 | g/liter |
| Thiamine HCl: | 0.8 | g/liter |
| Vitamin E: | 0.0188 | g/liter |
| Vitamin B12: | 0.17 | g/liter |
| Choline Chloride: | 2.51 | g/liter |
| Ethanolamine HCl: | 0.22 | g/liter |
| i-Inositol: | 3.66 | g/liter |
| Thymidine: | 0.39 | g/liter |
| Putrescine 2HCl: | 0.02 | g/liter |
| Progesterone: | 0.00075 | g/liter |
| D-Calcium Pantothenate: | 1.19 | g/liter |

In an embodiment of the invention, about 20 ml vitamin/salt feed is added per liter of culture. In an embodiment of the invention, the method further comprises harvesting the culture medium from the cells; wherein said protein is secreted from said cells into the medium. For example, in an embodiment of the invention, the culture medium is harvested from the cells when viability of the cells is below about 60%. The culture medium can be harvested from the cells, for example, by centrifuging the medium and/or depth filtering the medium and/or filtering the medium through a 0.2 micron filter. The protein expressed using the method of the invention, can be any protein, for example, an antibody or antigen-binding fragment thereof, e.g., that binds specifically to IGF1 R, e.g., wherein the antibody or fragment comprises a heavy immunoglobulin chain comprising the amino acid sequence set forth in a member selected from the group consisting of SEQ ID NOs: 1, 3,-6, 13, 21 and 26; or a mature fragment thereof or one or more CDRs therefrom;
and a light immunoglobulin chain comprising an amino acid sequence selected from the group consisting of SEQ ID NOs: 2, 7-9, 17 and 25, or a mature fragment thereof or one or more CDRs therefrom; for example, wherein the antibody or fragment is a full antibody that comprises a light chain immunoglobulin comprising amino acids 20-128 of SEQ ID NO: 3, 4, 5, or 6; and a heavy chain immunoglobulin comprising amino acids 20-137 of SEQ ID NO: 8 or 9, e.g., wherein the light chain immunoglobulin is linked to a kappa constant immunoglobulin chain and the heavy chain immunoglobulin is linked to a gamma-1 constant immunoglobulin chain. In an embodiment of the invention, the initial mammalian cell growth medium to which the supplements are added comprises HEPES, sodium bicarbonate buffers, inorganic salts, non-essential amino acids, recombinant human insulin, trace elements and surfactants; and does not comprise L-glutamine, antibiotics, antimycotics or animal-derived components.

The present invention includes within its scope a method for producing an antibody (e.g., monoclonal, recombinant and/or fully human) comprising inoculating an initial mammalian cell growth medium, pre-warmed to about 37° C.; which medium comprises HEPES, sodium bicarbonate buffers, inorganic salts, non-essential amino acids, recombinant human insulin, trace elements and surfactants; and which does not comprise L-glutamine, antibiotics, antimycotics or animal-derived components; with CHO DXB11 host cells expressing the antibody light chain immunoglobulin and heavy chain immunoglobulin, to a cell density of about 2.5−5×10⁵ cells/ml; and, adding the following supplements to the medium before, simultaneously with or immediately after said inoculation: soy hydrolysate to a final concentration of about 10 g/liter;

and, optionally, amino acids to about the following final concentrations (not including the quantity of said amino acids from other sources such as the initial mammalian cell growth medium):

| | | |
|---|---|---|
| L-arginine: | 126.4 | mg/liter |
| L-cystine: | 34 | mg/liter |
| L-histidine: | 42 | mg/liter |
| L-isoleucine: | 52 | mg/liter |
| L-leucine: | 52 | mg/liter |
| L-lysine: | 72 | mg/liter |
| L-Methionine: | 15.2 | mg/liter |
| L-phenylalanine: | 33 | mg/liter |
| L-threonine: | 47.6 | mg/liter |
| L-tryptophan: | 10.2 | mg/liter |
| L-tyrosine: | 36 | mg/liter |
| L-valine: | 46.8 | mg/liter |
| L-alanine: | 8.9 | mg/liter |
| L-asparagine: | 30 | mg/liter |
| L-aspartic acid: | 26.6 | mg/liter |
| L-glutamic acid: | 29.4 | mg/liter |
| Glycine: | 15 | mg/liter |
| L-proline: | 23 | mg/liter |
| L-serine: | 21 | mg/liter; |

and, when viable cell density reaches over about 1.2×10⁶ cells/ml, adding supplement feeds wherein the components from said supplement feeds (not including the quantity of each component from other feeds) reach an approximate final culture concentration set forth below:

| | | |
|---|---|---|
| Sodium selenite: | 0.01426 | mg/liter |
| Adenine sulfate: | 1.632 | mg/liter |
| Adenosine: | 17.6 | mg/liter |
| Cytidine: | 17.6 | mg/liter |
| Guanosine: | 17.6 | mg/liter |
| Uridine: | 17.6 | mg/liter |
| Hypoxanthine: | 11.8 | mg/liter |
| L-citrulline: | 12.6 | mg/liter |
| L-ornithine-HCl: | 25.6 | mg/liter |
| Biotin: | 0.28 | mg/liter |
| Flavin Adenine Dinucleotide: | 0.05 | mg/liter |
| Folic Acid: | 4.6 | mg/liter |
| Lipoic Acid: | 0.52 | mg/liter |
| Niacin: | 31.4 | mg/liter |
| Pyridoxine HCl: | 3 | mg/liter |
| Riboflavin: | 1.86 | mg/liter |
| Thiamine HCl: | 16 | mg/liter |
| Vitamin E: | 0.376 | mg/liter |
| Vitamin B12: | 3.4 | mg/liter |
| Choline Chloride: | 50.2 | mg/liter |
| Ethanolamine HCl: | 4.4 | mg/liter |
| i-Inositol: | 73.2 | mg/liter |
| Thymidine: | 7.8 | mg/liter |
| Putrescine 2HCl: | 0.4 | mg/liter |
| Progesterone: | 0.015 | mg/liter |
| D-Calcium Pantothenate: | 23.8 | mg/liter |
| L-asparagine: | 812 | mg/liter |
| L-proline | 216 | mg/liter |
| L-isoleucine | 370 | mg/liter |
| L-cysteine-HCl | 224 | mg/liter |
| L-leucine | 332 | mg/liter |
| L-threonine | 164 | mg/liter |
| L-tyrosine | 198 | mg/liter |
| L-arginine | 186 | mg/liter |
| L-aspartic acid | 71 | mg/liter |
| L-glutamic acid | 126 | mg/liter |
| Glycine | 57 | mg/liter |
| L-histidine | 125 | mg/liter |
| L-methionine | 132 | mg/liter |
| L-tryptophan | 99 | mg/liter |
| L-lysine | 293 | mg/liter |
| L-phenylalanine | 174 | mg/liter |
| L-valine | 262 | mg/liter |
| L-serine: | 260 | mg/liter |
| Sodium phosphate monobasic: | 288.2 | mg/liter |
| Zinc sulfate: | 1.08 | mg/liter |
| Cupric sulfate: | 0.0032 | mg/liter |
| Ammonium vanadate: | 0.00078 | mg/liter |
| Cobalt chloride: | 0.0025 | mg/liter |
| Nickel dichloride hexahydrate: | 0.0004 | mg/liter |
| Sodium molybdate dehydrate: | 0.00016 | mg/liter; |

and, during cell growth, adding glucose to the medium when glucose levels fall below about 1.5 g/liter and adding L-glutamine when L-glutamine levels fall below about 150 mg/liter; and during cell growth maintaining O₂ concentration at about 60%; pH at about 6.8±0.02 and temperature at about 36.5° C.±0.5° C. In an embodiment of the invention, the antibody comprises a heavy immunoglobulin chain comprising the amino acid sequence set forth in a member selected from the group consisting of SEQ ID NOs: 1, 3,-6, 13, 21 and 26; or a mature fragment thereof or one or more CDRs therefrom;
and a light immunoglobulin chain comprising an amino acid sequence selected from the group consisting of SEQ ID NOs: 2, 7-9, 17 and 25, or a mature fragment thereof or one or more CDRs therefrom, e.g., wherein the antibody comprises a light chain immunoglobulin comprising amino acids 20-128 of SEQ ID NO: 3, 4, 5, or 6; and a heavy chain immunoglobulin comprising amino acids 20-137 of SEQ ID NO: 8 or 9. For example, in an embodiment of the invention, the light chain immunoglobulin is linked to a kappa constant immunoglobulin chain and the heavy chain immunoglobulin is linked to a gamma-1 constant immunoglobulin chain. The method optionally further comprises recovering the culture medium from the cells by disk-stack centrifuging the medium, depth filtering the medium and filtering the medium through a filter with a 0.2 micron pore size and, optionally, the further step of purifying the immunoglobulin chains from the medium by column chromatographic fractionation.

The present invention also provides an aqueous liquid cell culture medium comprising:

about 10 g/liter soy hydrolysate,
about 1.5 g/liter glucose,
about 150 mg/liter L-glutamine,
pH of about 6.8±0.02,

### HEPES,

Sodium bicarbonate buffers,
Inorganic salts,
Non-essential amino acids,
Recombinant human insulin,
Trace elements,

### Surfactants,

an amino acid feed wherein the concentration of the components added by said amino acid feed are approximately those set forth below:

| | | |
|---|---|---|
| L-arginine: | 126.4 | mg/liter |
| L-cystine: | 34 | mg/liter |
| L-histidine: | 42 | mg/liter |
| L-isoleucine: | 52 | mg/liter |
| L-leucine: | 52 | mg/liter |
| L-lysine: | 72 | mg/liter |
| L-Methionine: | 15.2 | mg/liter |
| L-phenylalanine: | 33 | mg/liter |
| L-threonine: | 47.6 | mg/liter |
| L-tryptophan: | 10.2 | mg/liter |
| L-tyrosine: | 36 | mg/liter |
| L-valine: | 46.8 | mg/liter |
| L-alanine: | 8.9 | mg/liter |
| L-asparagine: | 30 | mg/liter |
| L-aspartic acid: | 26.6 | mg/liter |
| L-glutamic acid: | 29.4 | mg/liter |
| Glycine: | 15 | mg/liter |
| L-proline: | 23 | mg/liter |
| L-serine: | 21 | mg/liter; |

wherein the medium does not comprise antibiotics, antimycotics or animal-derived components. Moreover the present invention provides a cell culture in which a cell expressing a immunoglobulins of interest have been grown.

For example, the present invention provides a liquid culture medium produced by a process comprising inoculating an initial mammalian cell growth medium, pre-warmed to about 37° C.; which medium comprises HEPES, sodium bicarbonate buffers, inorganic salts, non-essential amino acids, recombinant human insulin, trace elements and surfactants; and which does not comprise L-glutamine, antibiotics, antimycotics or animal-derived components; with CHO DXB11 host cells expressing the antibody light chain immunoglobulin and heavy chain immunoglobulin, to a cell density of about 2.5−5×10⁵ cells/ml; and, adding the following supplements to the medium before, simultaneously with or immediately after said inoculation:

soy hydrolysate to a final concentration of about 10 g/liter;
and, optionally, an amino acid feed wherein the concentration of the components added by said amino acid feed are approximately those set forth below:

| | | |
|---|---|---|
| L-arginine: | 126.4 | mg/liter |
| L-cystine: | 34 | mg/liter |
| L-histidine: | 42 | mg/liter |
| L-isoleucine: | 52 | mg/liter |
| L-leucine: | 52 | mg/liter |
| L-lysine: | 72 | mg/liter |
| L-Methionine: | 15.2 | mg/liter |
| L-phenylalanine: | 33 | mg/liter |
| L-threonine: | 47.6 | mg/liter |
| L-tryptophan: | 10.2 | mg/liter |
| L-tyrosine: | 36 | mg/liter |
| L-valine: | 46.8 | mg/liter |
| L-alanine: | 8.9 | mg/liter |
| L-asparagine: | 30 | mg/liter |
| L-aspartic acid: | 26.6 | mg/liter |
| L-glutamic acid: | 29.4 | mg/liter |
| Glycine: | 15 | mg/liter |
| L-proline: | 23 | mg/liter |
| L-serine: | 21 | mg/liter; |

and, when viable cell density reaches over about 1.2×10⁶ cells/ml, adding supplement feeds wherein the concentration of the components added by said supplement feeds are approximately those set forth below:

| | | |
|---|---|---|
| Sodium selenite: | 0.01426 | mg/liter |
| Adenine sulfate: | 1.632 | mg/liter |
| Adenosine: | 17.6 | mg/liter |
| Cytidine: | 17.6 | mg/liter |
| Guanosine: | 17.6 | mg/liter |
| Uridine: | 17.6 | mg/liter |
| Hypoxanthine: | 11.8 | mg/liter |
| L-citrulline: | 12.6 | mg/liter |
| L-ornithine-HCl: | 25.6 | mg/liter |
| Biotin: | 0.28 | mg/liter |
| Flavin Adenine Dinucleotide: | 0.05 | mg/liter |
| Folic Acid: | 4.6 | mg/liter |
| Lipoic Acid: | 0.52 | mg/liter |
| Niacin: | 31.4 | mg/liter |
| Pyridoxine HCl: | 3 | mg/liter |
| Riboflavin: | 1.86 | mg/liter |
| Thiamine HCl: | 16 | mg/liter |
| Vitamin E: | 0.376 | mg/liter |
| Vitamin B12: | 3.4 | mg/liter |
| Choline Chloride: | 50.2 | mg/liter |
| Ethanolamine HCl: | 4.4 | mg/liter |
| i-Inositol: | 73.2 | mg/liter |
| Thymidine: | 7.8 | mg/liter |
| Putrescine 2HCl: | 0.4 | mg/liter |
| Progesterone: | 0.015 | mg/liter |
| D-Calcium Pantothenate: | 23.8 | mg/liter |
| L-asparagine: | 812 | mg/liter |
| L-proline | 216 | mg/liter |
| L-isoleucine | 370 | mg/liter |
| L-cysteine-HCl | 224 | mg/liter |
| L-leucine | 332 | mg/liter |
| L-threonine | 164 | mg/liter |
| L-tyrosine | 198 | mg/liter |
| L-arginine | 186 | mg/liter |
| L-aspartic acid | 71 | mg/liter |
| L-glutamic acid | 126 | mg/liter |
| Glycine | 57 | mg/liter |
| L-histidine | 125 | mg/liter |
| L-methionine | 132 | mg/liter |
| L-tryptophan | 99 | mg/liter |
| L-lysine | 293 | mg/liter |
| L-phenylalanine | 174 | mg/liter |
| L-valine | 262 | mg/liter |
| L-serine: | 260 | mg/liter |
| Sodium phosphate monobasic: | 288.2 | mg/liter |
| Zinc sulfate: | 1.08 | mg/liter |
| Cupric sulfate: | 0.0032 | mg/liter |
| Ammonium vanadate: | 0.00078 | mg/liter |
| Cobalt chloride: | 0.0025 | mg/liter |
| Nickel dichloride hexahydrate: | 0.0004 | mg/liter |
| Sodium molybdate dehydrate: | 0.00016 | mg/liter; |

and, during cell growth, adding glucose to the medium when glucose levels fall below about 1.5 g/liter and adding L-glutamine when L-glutamine levels fall below about 150 mg/liter; and during cell growth maintaining O₂ concentration at about 60%; pH at about 6.8±0.02 and temperature at about 36.5° C.±0.5° C.; wherein cell viability is about 60% or lower. In an embodiment of the invention, the culture medium includes host cells which comprise a vector encoding an antibody or antigen-binding fragment thereof; optionally, wherein host cell secreted antibody or fragment is in the medium. For example, in an embodiment of the invention, the immunoglobulins form an antibody or antigen-binding fragment thereof that binds specifically to IGF1 R, e.g., wherein the antibody or fragment comprises a heavy immunoglobulin chain comprising the amino acid sequence set forth in a member selected from the group consisting of SEQ ID NOs: 1, 3,-6, 13, 21 and 26; or a mature fragment thereof or one or more CDRs therefrom; and/or a light immunoglobulin chain comprising an amino acid sequence selected from the group consisting of SEQ ID NOs: 2, 7-9, 17 and 25, or a mature fragment thereof or one or more CDRs therefrom, e.g., wherein the antibody or fragment comprises a light chain immunoglobulin comprising amino acids 20-128 of SEQ ID NO: 3, 4, 5, or 6; and a heavy chain immunoglobulin comprising amino acids 20-137 of SEQ ID NO: 8 or 9. In an embodiment of the invention, the light chain immunoglobulin is linked to a kappa constant immunoglobulin chain and the heavy chain immunoglobulin is linked to a gamma-1 constant immunoglobulin chain. For example, wherein the host cells comprise a vector encoding immunoglobulins of an antibody or antigen-binding fragment thereof wherein the antibody or fragment is secreted into the medium. In an embodiment of the invention, the host cell viability is about 60% or lower and/or wherein cell growth has proceeded for about 14-24 days (e.g., about 14 days, about 15 days, about 16 days, about 17 days, about 18 days, about 19 days, about 20 days, about 21 days, about 22 days, about 23 days or about 24 days), e.g., by centrifuging the medium and cells and removing the cells from the medium, e.g., wherein the medium is also depth filtered and/or filtered through a 0.2 micron filter, for example, after centrifugation. Any medium comprising the characteristics of the medium generated as set forth above forms part of the present invention regardless of the process by which the medium was actually generated.

The present invention also provides a vessel (e.g., flask, a bioreactor, a tank bioreactor, a bag bioreactor or a disposable bioreactor, stirred tank bioreactor, a bubble column bioreactor, an air lift bioreactor, a fluidized bed bioreactor, or a packed bed bioreactor) comprising any culture medium of the invention.

### DETAILED DESCRIPTION OF THE INVENTION

The present application includes processes for antibody production (e.g., anti-IGF1R) from mammalian cells such as CHO (Chinese Hamster Ovary) cells. The processes are run in a variety of bioreactor culture systems, including stirred tank bioreactors, bag and disposable bioreactors, and shake flasks. The “level 3” production process yields, on average, a titer of about 1.2 g/L of antibody and a culture specific productivity of about 22 pg/cell/day. The “enhanced” process yields, on average, a titer of about 2.3 g/L antibody, with a specific productivity of about 30 pg/cell/day. Both processes involve the addition of specific supplements to the production bioreactor culture either prior to inoculation or as in-process feeds to increase culture specific productivity, titer, biomass, and culture viability. For the level 3 process, three supplements are added-wheat and/or soy hydrolysates (hydrolysate feeds) are added (e.g., as 200 g/L (aq) solutions) to increase culture specific productivity. Either both hydrolysates or the soy hydrolysate feed by itself are typically added to the culture. Two concentrated feed solutions of amino acids (amino acid feeds) are added to elevate culture biomass and increase process titer. A concentrated vitamin and salt solution (vitamin/salt feed) is added to stabilize culture viability and increase biomass. The vitamin feed solution includes, for example, biotin, progesterone, inositol, nucleic acids, citrulline, hypoxanthine, lipoic acid, riboflavin, thiamine, choline, ethanolamine, folic acid, flavin, and vitamin B12.

For the enhanced process, the feed solutions are added, along with an additional concentrated nutrient solution (nutrient feed). The “nutrient feed” includes, for example, amino acids, zinc sulfate, cupric sulfate, ammonium vanadate, cobalt chloride, nickel dichloride, tin chloride, and manganese chloride. It can be added prior to inoculation or as an in-process feed to improve process titer and increase viability.

In addition, an in-process osmolality shift (to 400-500 mOsm) is optionally employed by addition of a concentrated salt solution, soy hydrolysate solution or carbon dioxide and sodium hydroxide (as part of the pH control for the bioreactor). The osmolality shift is employed in order to increase culture specific productivity and improve harvest viability.

Furthermore, an in-process temperature downshift, from about 36° C. to 37° C. to 33-35° C., is optionally used to stabilize culture viability at the high viable cell counts.

For the purposes of the present invention, the term “in-process” refers to an event occurring during growth of a production cell culture following an initial cell inoculation (e.g., of cells expanded, for example, for a master cell bank or working cell bank).

A “production cell culture” refers to the cell culture from which a final product, such as an antibody (e.g., anti-IGF1R antibody) is to be isolated.

An “expansion cell culture” refers to cells or a culture of cells to be used for initial inoculation of a production cell culture.

An “nX” stock solution of supplements (wherein n is a number, such as 50), indicates that the stock solution is diluted by 1/n when added to the culture. For example, a 50× stock solution is normally diluted 1/50 when added to a culture.

### Feeds

The processes of the present invention include steps wherein various feeds are added to an initial mammalian cell growth medium. These feeds include hydrolysate feed, vitamin/salt feed, amino acid feed and nutrient feed. Depending on the type of cell culture process being used, the level 3 process or the enhanced process, the feeds may be added at different points.

The “initial mammalian cell growth medium” can be any of several types of aqueous mediums known in the art; and the meaning of this term would be readily known by any practitioner of ordinary skill in the art. Examples include EX-CELL ACF CHO medium (Sigma-Aldrich (St. Louis, Mo.); discussed further below), DMEM, DMEM/F-12, F-10 Nutrient Mixture, RPMI Medium 1640, F-12 Nutrient Mixture, Medium 199, Eagle's MEM, RPMI, 293 media, and Iscove's Media. For example, Eagle's minimal essential medium (MEM) comprises L-Arginine hydrochloride (126 mg/l), L-Cystine 2HCl (31 mg/l), L-Histidine hydrochloride-H₂O (42 mg/l), L-Isoleucine (52 mg/l), L-Leucine (52 mg/l), L-Lysine hydrochloride (73 mg/l), L-Methionine (15 mg/l), L-Phenylalanine (32 mg/l), L-Threonine (48 mg/l), L-Tryptophan (10 mg/l), L-Tyrosine disodium salt dehydrate (52 mg/l), L-Valine (46 mg/l), Choline chloride (1 mg/l), D-Calcium pantothenate (1 mg/l), Folic Acid (1 mg/l), Niacinamide (1 mg/l), Pyridoxal hydrochloride (1 mg/l), Riboflavin (0.1 mg/l), Thiamine hydrochloride (1 mg/l), i-Inositol (2 mg/l), Calcium Chloride (CaCl₂) (anhyd.) (200 mg/l), Magnesium Sulfate (MgSO₄) (anhyd.) (97.67 mg/l), Potassium Chloride (KCl) (400 mg/l), Sodium Bicarbonate (NaHCO₃) (2200 mg/l), Sodium Chloride (NaCl) (6800 mg/l), Sodium Phosphate monobasic (NaH₂PO₄—H₂O) (140 mg/l), D-Glucose (Dextrose) (1000 mg/l) and Phenol Red (10 mg/l).

Modified Eagle Medium (MEM) (2×) comprises L-Arginine hydrochloride (504 mg/l), L-Cystine (96 mg/l), L-Glutamine (870 mg/l), L-Histidine hydrochloride-H₂O (168 mg/l), L-Isoleucine (208 mg/l), L-Leucine (208 mg/l), L-Lysine hydrochloride (290 mg/l), L-Methionine (60 mg/l), L-Phenylalanine (128 mg/l), L-Threonine (192 mg/l), L-Tryptophan (40 mg/l), L-Tyrosine disodium salt dehydrate (208 mg/l), L-Valine (155 mg/l), Choline chloride (4 mg/l), D-Calcium pantothenate (4 mg/l), Folic Acid (4 mg/l), Niacinamide (4 mg/l), Pyridoxal hydrochloride (4 mg/l), Riboflavin (0.4 mg/l), Thiamine hydrochloride (4 mg/l), i-Inositol (8 mg/l), Calcium Chloride (CaCl₂) (anhyd.) (285 mg/l), Ferric Nitrate (Fe(NO₃)₃″9H₂O) (1 mg/l), Magnesium Sulfate (MgSO₄) (anhyd.) (195 mg/l), Potassium Chloride (KCl) (800 mg/l), Sodium Bicarbonate (NaHCO₃) (8400 mg/l), Sodium Chloride (NaCl) (12800 mg/l), Sodium Phosphate monobasic (NaH₂PO₄-H₂O) (250 mg/l) and D-Glucose (Dextrose) (9000 mg/l).

RPMI Medium 1640 (1×) comprises Glycine (10 mg/l), L-Arginine (200 mg/l), L-Asparagine (50 mg/l), L-Aspartic acid (20 mg/l), L-Cystine 2HCl (65 mg/l), L-Glutamic Acid (20 mg/l), L-Glutamine (300 mg/l), L-Histidine (15 mg/l), L-Hydroxyproline (20 mg/l), L-Isoleucine (50 mg/l), L-Leucine (50 mg/l), L-Lysine hydrochloride (40 mg/l), L-Methionine (15 mg/l), L-Phenylalanine (15 mg/l), L-Proline (20 mg/l), L-Serine (30 mg/l), L-Threonine (20 mg/l), L-Tryptophan (5 mg/l), L-Tyrosine disodium salt dehydrate (29 mg/l), L-Valine (20), Biotin (0.2 mg/l), Choline chloride (3 mg/l), D-Calcium pantothenate (0.25 mg/l), Folic Acid (1 mg/l), Niacinamide (1 mg/l), Para-Aminobenzoic Acid (1 mg/l), Pyridoxine hydrochloride (1 mg/l), Riboflavin (0.2 mg/l), Thiamine hydrochloride (1 mg/l), Vitamin B12 (0.005 mg/l), i-Inositol (35 mg/l), Calcium nitrate (Ca(NO₃)₂ 4H2O) (100 mg/l), Magnesium Sulfate (MgSO₄) (anhyd.) (48.84 mg/l), Potassium Chloride (KCl) (400 mg/l), Sodium Bicarbonate (NaHCO₃) (2000 mg/l), Sodium Chloride (NaCl) (6000 mg/l), Sodium Phosphate dibasic (Na₂HPO₄) anhydrous (800 mg/l), D-Glucose (Dextrose) (2000 mg/l) and Glutathione (reduced) (1 mg/l).

Generally, for the purposes of the present invention a “hydrolysate feed” includes wheat and/or soy hydrolysates. Generally, a soy or wheat hydrolysate is the product of an enzymatic digest of soy or wheat and can be purchased commercially. Typically, the hydrolysate is in cell culture grade water and is sterile. In an embodiment of the invention, the hydrolysate is a stock solution at 200 g/liter. In an embodiment of the invention, the hydrolysate is added to the culture medium to reach a final concentration of about 10 g/liter. In an embodiment of the invention, when using either the level 3 process or the enhanced process, the hydrolysate is added to the culture medium either initially, before, with or immediately after inoculation or at about 3 days after inoculation or when viable cell density reaches over about 1×10⁶ cells/ml.

“Viable cell density” refers to the concentration of cells in the medium being analyzed (e.g., cells/ml) which are viable, e.g., capable of growth and replication (e.g., when used to inoculate a liquid culture or a solid culture medium) or capable of excluding a dye such as tryptan blue, eosin or propidiumin in a dye exclusion assay. Such assays are commonly known in the art.

Generally, for the purposes of the present invention, a “vitamin/salt feed” includes:

| | |
|---|---|
| Sodium selenite | e.g., at a concentration of about 7.13 × 10⁻⁴ g/liter |
| Adenine sulfate | e.g., at a concentration of about 0.0816 g/liter |
| Adenosine | e.g., at a concentration of about 0.88 g/liter |
| Cytidine | e.g., at a concentration of about 0.88 g/liter |
| Guanosine | e.g., at a concentration of about 0.88 g/liter |
| Uridine | e.g., at a concentration of about 0.88 g/liter |
| Hypoxanthine | e.g., at a concentration of about 0.59 g/liter |
| L-citrulline | e.g., at a concentration of about 0.63 g/liter |
| L-ornithine-HCl | e.g., at a concentration of about 1.28 g/liter |
| Biotin | e.g., at a concentration of about 0.014 g/liter |
| Flavin Adenine | e.g., at a concentration of about 0.0025 g/liter |
| Dinucleotide | |
| Folic Acid | e.g., at a concentration of about 0.23 g/liter |
| Lipoic Acid | e.g., at a concentration of about 0.026 g/liter |
| Niacin | e.g., at a concentration of about 1.57 g/liter |
| Pyridoxine HCl | e.g., at a concentration of about 0.15 g/liter |
| Riboflavin | e.g., at a concentration of about 0.093 g/liter |
| Thiamine HCl | e.g., at a concentration of about 0.8 g/liter |
| Vitamin E | e.g., at a concentration of about 0.0188 g/liter |
| Vitamin B12 | e.g., at a concentration of about 0.17 g/liter |
| Choline Chloride | e.g., at a concentration of about 2.51 g/liter |
| Ethanolamine HCl | e.g., at a concentration of about 0.22 g/liter |
| i-Inositol | e.g., at a concentration of about 3.66 g/liter |
| Thymidine | e.g., at a concentration of about 0.39 g/liter |
| Putrescine 2HCl | e.g., at a concentration of about 0.02 g/liter |
| Progesterone | e.g., at a concentration of about 0.00075 g/liter; and |
| D-Calcium | e.g., at a concentration of about 1.19 g/liter |
| Pantothenate | |

In an embodiment of the invention, the vitamin/salt feed is a 50× stock solution. In an embodiment of the invention, the vitamin/salt feed is added to the culture medium to reach a final concentration of about 20 ml/liter. When employing the level 3 process, the vitamin/salt feed is added to the culture between days 3 and 5, post-inoculation, or when viable cell density reaches over about 1×10⁶ cells/ml. In an embodiment of the invention, when employing the enhanced process, the vitamin/salt feed is added to the culture between days 3 and 5, post-inoculation, or when viable cell density reaches over about 1.2×10⁶ cells/ml.

Generally, for the purposes of the present invention, an “amino acid feed” includes:

| | |
|---|---|
| L-arginine | e.g., at a concentration of about 6.32 g/liter |
| L-cystine | e.g., at a concentration of about 1.7 g/liter |
| L-histidine | e.g., at a concentration of about 2.1 g/liter |
| L-isoleucine | e.g., at a concentration of about 2.6 g/liter |
| L-leucine | e.g., at a concentration of about 2.6 g/liter |
| L-lysine | e.g., at a concentration of about 3.6 g/liter |
| L-Methionine | e.g., at a concentration of about 0.76 g/liter |
| L-phenylalanine | e.g., at a concentration of about 1.65 g/liter |
| L-threonine | e.g., at a concentration of about 2.38 g/liter |
| L-tryptophan | e.g., at a concentration of about 0.51 g/liter |
| L-tyrosine | e.g., at a concentration of about 1.8 g/liter |
| L-valine | e.g., at a concentration of about 2.34 g/liter |
| L-alanine | e.g., at a concentration of about 0.89 g/liter |
| L-asparagine | e.g., at a concentration of about 1.5 g/liter |
| L-aspartic acid | e.g., at a concentration of about 1.33 g/liter |
| L-glutamic acid | e.g., at a concentration of about 1.47 g/liter |
| Glycine | e.g., at a concentration of about 0.75 g/liter |
| L-proline | e.g., at a concentration of about 1.15 g/liter; and |
| L-serine | e.g., at a concentration of about 1.05 g/liter |

In an embodiment of the invention, two separate amino acid feed stock solutions are prepared: a 100× stock solution including L-alanine, L-asparagine, L-aspartic acid, L-glutamic acid, glycine, L-proline and L-serine at the concentrations set forth above; and and a 50× solution including L-arginine, L-cystine, L-histidine, L-isoleucine, L-leucine, L-lysine, L-Methionine, L-phenylalanine, L-threonine, L-tryptophan, L-tyrosine, and L-valine at the concentrations set forth above. These stocks can be made and added separately to the culture medium. In an embodiment of the invention, the amino acid stock solution is added to the initial medium at day 0, before, with or immediately after cell inoculation.

Generally, for the purposes of the present invention, a “nutrient feed” includes:

| | |
|---|---|
| L-asparagine: | e.g., at a concentration of about 40.6 g/liter |
| L-proline | e.g., at a concentration of about 10.81 |
| L-isoleucine | e.g., at a concentration of about 18.53 |
| L-cysteine-HCl | e.g., at a concentration of about 11.19 |
| L-leucine | e.g., at a concentration of about 16.58 |
| L-threonine | e.g., at a concentration of about 8.2 |
| L-tyrosine | e.g., at a concentration of about 9.9 |
| L-arginine | e.g., at a concentration of about 9.29 |
| L-aspartic acid | e.g., at a concentration of about 3.56 |
| L-glutamic acid | e.g., at a concentration of about 6.28 |
| Glycine | e.g., at a concentration of about 2.83 |
| L-histidine | e.g., at a concentration of about 6.23 |
| L-methionine | e.g., at a concentration of about 6.58 |
| L-tryptophan | e.g., at a concentration of about 4.93 |
| L-lysine | e.g., at a concentration of about 14.66 |
| L-phenylalanine | e.g., at a concentration of about 8.64 |
| L-valine | e.g., at a concentration of about 13.08 g/liter |
| L-serine: | e.g., at a concentration of about 13 g/liter |
| Sodium phosphate | e.g., at a concentration of about 14.41 g/liter |
| monobasic: | |
| Zinc sulfate: | e.g., at a concentration of about 0.054 g/liter |
| Cupric sulfate: | e.g., at a concentration of about 0.00016 g/liter |
| Ammonium vanadate: | e.g., at a concentration of about 0.000039 g/liter |
| Cobalt chloride: | e.g., at a concentration of about 0.000125 g/liter |
| Nickel dichloride | e.g., at a concentration of about 0.00002 g/liter |
| hexahydrate: | |
| Sodium molybdate | e.g., at a concentration of about 0.000008 g/liter |
| dehydrate: | |
| Tin chloride | e.g., at a concentration of about 0.000004 g/liter |
| dehydrate: | |
| Manganese chloride: | e.g., at a concentration of about 0.000015 g/liter |
| tetrahydrate: | |

In an embodiment of the invention, the nutrient feed is a 50× stock solution. In an embodiment of the invention, the nutrient feed is added to the culture medium to reach a final concentration of about 20 ml/liter. When employing the enhanced process, the nutrient feed is added to the culture between days 3 and 5, post-inoculation, or when viable cell density reaches about 1.2×10⁶ cells/ml.

Furthermore, in an embodiment of the invention, when employing either the level 3 or enhanced process, glucose (from a 2.5 M stock solution) and L-glutamine (from a 0.2 M stock solution) are added to the culture medium at any point, e.g., when the concentration of the nutrients fall below 1.5 g/liter glucose and 150 mg/liter L-glutamine.

### Osmolality and Temperature Shift

The present invention also includes processes wherein the osmolality and/or the temperature of the culture is optionally shifted. The osmolality or temperature shift may be done at any point in-process.

The osmolality shift has been shown to increase culture specific productivity as well as cell viability. Typically, the initial mammalian cell growth medium has a starting osmolality of about 300 mOsm. The “osmolality shift” of the present invention, however, includes shifting the culture osmolality to from about 400 mOsM to about 500 mOsm.

Osmolality is a measure of the osmoles of solute per kilogram of solvent. Osmolality can be measured using an osmometer which measures colligative properties, such as freezing-point depression, vapor pressure, or boiling-point elevation.

Osmolality of a cell culture may be shifted by any of several means. For example, a concentrated salt solution (e.g., including 5M NaCl salts stock, 8-12 mL/L added), soy hydrolysate solution (200 g/L stock, 50-80 mL/L added), or carbon dioxide may be added. In an embodiment of the invention, adding the nutrient feed to the medium shifts the osmolality.

In an embodiment of the invention, the temperature of the culture is optionally shifted, e.g., in a step change, from about 36.5° C. (+0.5° C.) to between about 33° C. and 35° C.

### Proteins

The present invention includes embodiments comprising methods for recombinantly producing proteins such as immunoglobulin chains. In an embodiment of the invention, the immunoglobulin comprises an anti-IGF1 R antibody (e.g., human antibody, humanized antibody, chimeric antibody) or antigen-binding fragment thereof, e.g., including an immunoglobulin light and/or heavy chain variable region, optionally linked with an immunoglobulin constant region.

For example, the present invention includes methods wherein a protein to be expressed (e.g., an anti-IGF1R antibody light chain or heavy chain immunoglobulin) is encoded by a polynucleotide in a plasmid vector, e.g., wherein the polynucleotide is operably linked to a promoter such as a CMV promoter. In an embodiment of the invention, the light and heavy chains are included in a single plasmid vector.

In an embodiment of the invention, the immunoglobulin chain encodes any of those set forth below; for example, any of the following immunoglobulin light and/or heavy chains and/or any of the CDRs thereof (e.g., all 3 from a single light or heavy chain). Dotted, underscored type encodes the signal peptide. Solid underscored type encodes the CDRs. Plain type encodes the framework regions. In an embodiment of the invention, the chains are expressed with the signal peptide which is cleaved upon secretion from the host cell to generate a mature fragment of the chain.

Compositions and processes for producing any of the following target immunoglobulin amino acid sequences or mature fragments thereof form part of the present invention.

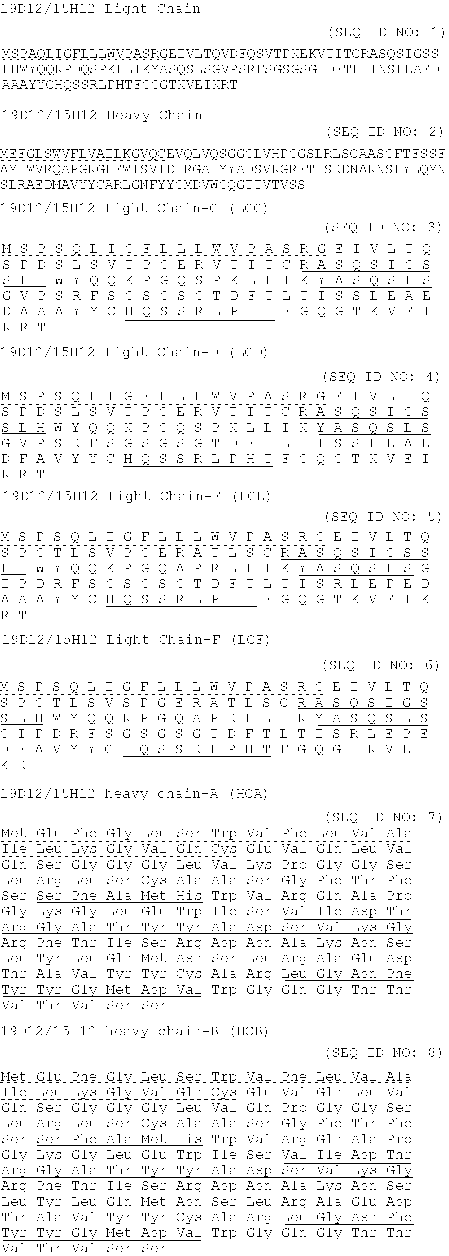

See international application publication no. WO2003/100008 which is incorporated herein by reference in its entirety.

2C6 heavy chain
(SEQ ID NO: 9)
MELGLSWIFLLAILKGVQC
EVQLVESGGGLVQPGRSLRLSCAASGFTFDDYAMHWVRQAPGKGLEWVS
GISWNSGSKGYVDSVKGRFTISRDNAKNSLYLQMNSLRAEDTALYYCAK
DIRIGVAASYYFGMDVWGHGTTVTVSS
2C6 CDR-H1:
(SEQ ID NO: 10)
GFTFDDYAMH
2C6 CDR-H2:
(SEQ ID NO: 11)
GISWNSGSKGYVDSVKG
2C6 CDR-H3:
(SEQ ID NO: 12)
DIRIGVAASYYFGMDV
2C6 Light chain
(SEQ ID NO: 13)
MDMRVPAQLLGLLLLWLPGARC
AIQLTQSPSSLSASVGDRVTITCRASQGISSVLAWYQQKPGKAPKLLIY
DASSLESGVPSRFSGSGSGTDFTLTISSLQPEDFATYYCQQFNSYPYT
FGQGTKLEIK
2C6 CDR-L1:
(SEQ ID NO: 14)
RASQGISSVLA
2C6 CDR-L2:
(SEQ ID NO: 15)
DASSLES
2C6 CDR-L3:
(SEQ ID NO: 16)
QQFNSYPYT
9H2 Heavy chain
(SEQ ID NO: 17)
MDWTWRILFLVAAATGAHS
QVQLVQSGAEVKKPGASVKVSCKASGYTFTSYVMHWVRQAPGQRLEWMG
WINAGNGNTKYSQKFQGRVTITRDTSASTVYMELSSLRSEDTAVYYCAR
GGMPVAGPGYFYYYGMDVWGQGTTVTVSS
9H2 CDR-H1:
(SEQ ID NO: 18)
GYTFTSYVMH
9H2 CDR-H2:
(SEQ ID NO: 19)
WINAGNGNTKYSQKFQG
9H2 CDR-H3:
(SEQ ID NO: 20)
GGMPVAGPGYFYYYGMDV
9H2 Light chain
(SEQ ID NO: 21)
METPAQLLFLLLLWLPDTTG
EIVLTQSPG1LSLSPGERATLSCRASQSVSRSYLAWYQQKPGQAPRLLIY
GASSRATGIPDRFSGSGSGTDFTLTISRLEPEDFAVYCCQQYGSSPWT
FGQGTKVEIKRT
9H2 CDR-L1:
(SEQ ID NO: 22)
RASQSVSRSYLA
9H2 CDR-L2:
(SEQ ID NO: 23)
GASSRAT
9H2 CDR-L3:
(SEQ ID NO: 24)
QQYGSSPWT
Heavy chain immunoglobulin variable region
# 1.0 sequence
(SEQ ID NO: 25)
E VQLLESGGGL VQPGGSLRLS CTASGFTFSS YAMNWVRQAP
GKGLEWVSAI SGSGGTTFYA DSVKGRFTIS RDNSRTTLYL
QMNSLRAEDT AVYYCAKDLG WSDSYYYYYG MDVWGQGTTV
TVSS;
Light chain immunoglobulin variable region
# 1.0 sequence
(SEQ ID NO: 26)
DIQMTQFP SSLSASVGDR VTITCRASQG IRNDLGWYQQ
KPGKAPKRLI YAASRLHRGV PSRFSGSGSG TEFTLTISSL
QPEDFATYYC LQHNSYPCSF GQGTKLEIKR;

Embodiments of the invention include those wherein the immunoglobulin is expressed, for example, in combination of any of those set forth herein (e.g., heavy chain Ig. # 1.0 and light chain 1 g. #1.0; or LCC and HCA; or LCF and HCA; or LCC and HCB). Pairing of the light and heavy chains can result in generation of an antibody or antigen-binding fragment thereof.

In an embodiment of the invention, the light chain is fused to an immunoglobulin constant chain, e.g., a kappa chain. In an embodiment of the invention, the heavy chain is fused to an immunoglobulin constant chain, e.g., a gamma-1, gamma-2, gamma-3 or gamma-4 chain.

Other proteins of interest that may be expressed using the methods and compositions of the present invention include receptors, ligands, cytokines, chemokines, growth factors, hormones and enzymes.

### Production Process and Materials

Vectors, such as plasmids, including a gene to be expressed by a process of the present invention may be introduced into a host cell by any of several methods known in the art. Transformation can be carried out, e.g., by the calcium phosphate precipitation method as described by Graham and Van der Eb, Virology, 52: 546 (1978). Other methods for introducing DNA into cells such as by nuclear injection or by protoplast fusion may also be used. Methods for transformation also include electroporation, liposomal transformation and DEAE-Dextran transformation.

Host cells are, in an embodiment of the invention, mammalian cells. For example, Chinese hamster ovary cells (CHO cells). A CHO-K1 cell is proline-requiring and is diploid for the dihydrofolate reductase (dhfr) gene. In an embodiment of the invention, the cell line is the DXB11 CHO cell line (Urlaub et al., (1983) Cell 33: 405-412). Other cell lines include, for example, HEK293.

Host cells comprising a gene to be expressed using a process of the present invention may be selected and screened to identify the clone with the requisite characteristics for expression of a target gene. In Chinese hamster ovary (CHO) cells, one common approach, to achieve maximal expression, involves the use of mutant cell lines and a gradual increase in the selection pressure over several months for a co-transfected selection marker such as dihydrofolate reductase (DHFR) (Kaufman et al., (1982) J. Mol. Biol. 159: 601-621; Schimke et al. (1982) Natl. Cancer Inst. Monogr. 60: 79-86). In order to achieve high production rates, a dihydrofolate reductase (DHFR) negative cell line (e.g., a CHO cell line) (Urlaub et al. (1980) Proc. Natl. Acad. Sci. USA 77: 4216-4220) is transformed with an expression vector containing a functional DHFR gene in combination with the target gene to be expressed. Amplification of the vector-inserted target genes occurs in response to addition of increasing amounts of the DHFR antagonist methotrexate (MTX) to the culture medium and clones or subpopulations carrying multiple copies of the recombinant genes are generated and can be selected (Wurm (1990) Biologicals 18:159-164). The gene amplification process typically takes several months until stable cell lines are obtained which show high target gene copy numbers and high production rates of the desired protein.

In an embodiment of the invention, a polynucleotide of the present invention is integrated into host cell (e.g., CHO, CHO-K1, CHO DXB11) chromosomal DNA or ectopic and autonomously replicating. In an embodiment of the invention, the polynucleotide of the present invention is present in the cell at several copies per cell (e.g., 2, 3, 4, 5, 6, 7, 8, 9, 10, 15, 20). Where an expression vector has been integrated into the genomic DNA of the host cell to improve stability, the copy number of the vector DNA, and, concomitantly, the amount of product which could be expressed, can be increased by selecting for cell lines in which the vector sequences have been amplified after integration into the DNA of the host cell. Integrated genes may be screened for the presence and relative amount of chromosomally incorporated DNA and corresponding mRNA and polypeptide synthesis by standard methods. For example, the presence of the desired integrate may be detected by standard procedures such as DNA sequencing, Southern blotting, Northern blotting and/or Western blotting.

Any of several cell culture mediums known in the art can be used to propagate cells expressing a target gene. Several commercially available culture mediums are available. If expressing a protein to be used therapeutically, animal-product-free media (e.g., serum-free media (SFM)) may be desirable. There are several methods known in the art by which cells may be adapted to growth in serum-free medium. For example, direct adaptation includes merely switching cells from serum-supplemented media to serum-free media. Sequential adaptation or weaning includes switching cells from a serum-supplemented medium into a serum-free medium in several steps (e.g., 25% SFM, 50% SFM, 75% SFM, then, 90% SFM for about 3 passages, then 100% SFM). Sequential adaptation tends to be less harsh on cells than direct adaptation. Generally, to adapt cells to serum-free media, the culture should be in mid-logarithmic phase, >90% viable and seeded at a higher initial cell inoculum than during direct adaptation. In an embodiment of the invention, the initial mammalian cell growth medium is EX-CELL ACF CHO medium which is commercially available from Sigma-Aldrich (St. Louis, Mo.). This culture is animal-component free, with HEPES (N-2-Hydroxyethylpiperazine-N′-2-ethanesulfonic acid), without L-glutamine, liquid, sterile-filtered and cell culture tested. The medium also includes inorganic salts, sodium bicarbonate buffers, essential and non-essential amino acids, vitamins, recombinant human insulin, plant hydrolysates, other organic compounds, trace elements, and surfactants. The medium also does not contain antibiotics, antimycotics or transferrin and also contains no animal-derived proteins or other components. Typically, for reconstitution of the medium, a practitioner should aseptically add 20-40 ml of 200 mM L-glutamine solution per liter of medium prior to use.

A cell line containing a host cell comprising a gene to be expressed using a process of the present invention may also be stored in a master cell bank (MCB) and/or working cell bank (WCB). Typically, when a cell line is to be used over many manufacturing cycles, a two-tiered cell banking system consisting of a master cell bank or master seed bank (MSB) and a working cell bank can be established. A cell line is established from a single host cell clone and this cell line is used to make-up the MCB. Generally, this MCB must be characterized and extensively tested for contaminants such as bacteria, fungi, viruses and mycoplasma. A sample of cells from the MCB can be expanded to form the WCB, which is characterized for cell viability prior to use in a manufacturing process. The cells in a MCB or WCB can be stored in vials, for example, at low temperature (e.g., 0° C. or lower, −20° C. or −80° C.).

Typically, the working cell bank includes cells from one vial of the master bank which have been grown for several passages before storage. In general, when future cells are needed, they are taken from the working cell bank; whereas, the master cell bank is used only when necessary, ensuring a stock of cells with a low passage number to avoid genetic variation within the cell culture.

The present invention provides two processes for growing cells and recombinantly producing a protein-the “level 3” process and the “enhanced process”. Both processes generate high levels of proteins of interest, however the enhanced process generates especially high levels.

In an embodiment of the invention, the level 3 process for producing a protein, such as an antibody (e.g., anti-IGF1R) comprises the steps:

1-expanding cells expressing the protein in a standard initial mammalian growth media (e.g., Sigma CHO medium with added L-glutamine (4 mM)).

This expansion can be done, for example, in shake flasks. In an embodiment of the invention, expansion occurs by growth to about 1-2×10⁶ cells/ml, dilution of a sample of those cells (e.g., to a density of about 2.5-5×10⁵ cells/ml) and, then, re-growth to about 1-2×10⁶ cells/ml, for about 10-30 passages.

2—inoculating an initial mammalian cell growth medium with the expanded cells from step (1), to a cell density of about 2.5-5×10⁵ cells/ml, and adding supplements to the medium. The supplements are wheat and/or soy hydrolysate, amino acid feed, vitamin/salt feed, glucose and L-glutamine.

The day of inoculation is “day 0”, the following day is “day 1”, the following day is “day 2”, and so on.

Soy or wheat hydrolysates are added, for example, either on day 0 or after viable cell density has reached over about 10⁶ cells/ml. In an embodiment of the invention, the hydrolysate(s) are simply added on day 3 when the viable cell density typically reaches 10⁶ cells/ml.

Amino acid feeds (discussed above) are added, for example, at day 0, e.g., to reach approximate final culture concentrations as set forth below (not including the concentration of any indicated component from other sources such as from the initial mammalian cell growth medium):

| | Final concentration in |
|---|---|
| Component | culture (mg/liter) |
| L-arginine | 126.4 |
| L-cystine | 34 |
| L-histidine | 42 |
| L-isoleucine | 52 |
| L-leucine | 52 |
| L-lysine | 72 |
| L-Methionine | 15.2 |
| L-phenylalanine | 33 |
| L-threonine | 47.6 |
| L-tryptophan | 10.2 |
| L-tyrosine | 36 |
| L-valine | 46.8 |
| L-alanine | 8.9 |
| L-asparagine | 30 |
| L-aspartic acid | 26.6 |
| L-glutamic acid | 29.4 |
| Glycine | 15 |
| L-proline | 23 |
| L-serine | 21 |

In an embodiment of the invention, amino acid feeds are not added to the medium.

Vitamin/salt feed solution (discussed above) is added, for example, between days 3 and 5 or when viable cell density reaches about 10⁶ cells/ml, e.g., to reach approximate final culture concentrations as set forth below (not including the concentration of any indicated component from other sources such as from the initial mammalian cell growth medium):

| | Final culture concentrations |
|---|---|
| Component | (mg/liter) |
| Sodium selenite | 0.01426 |
| Adenine sulfate | 1.632 |
| Adenosine | 17.6 |
| Cytidine | 17.6 |
| Guanosine | 17.6 |
| Uridine | 17.6 |
| Hypoxanthine | 11.8 |
| L-citrulline | 12.6 |
| L-ornithine-HCl | 25.6 |
| Biotin | 0.28 |
| Flavin Adenine Dinucleotide | 0.05 |
| Folic Acid | 4.6 |
| Lipoic Acid | 0.52 |
| Niacin | 31.4 |
| Pyridoxine HCl | 3 |
| Riboflavin | 1.86 |
| Thiamine HCl | 16 |
| Vitamin E | 0.376 |
| Vitamin B12 | 3.4 |
| Choline Chloride | 50.2 |
| Ethanolamine HCl | 4.4 |
| i-Inositol | 73.2 |
| Thymidine | 7.8 |
| Putrescine 2HCl | 0.4 |
| Progesterone | 0.015 |
| D-Calcium Pantothenate | 23.8 |

Glucose is added, for example, when the glucose concentration in the culture medium falls below about 1.5 g/liter and L-glutamine is added, for example, when the glutamine concentration in the culture medium falls below about 150 mg/liter.

3—Optionally harvesting the cells from the production cell culture medium, e.g., when viability is below 60%, by removing the cells from the culture medium (e.g., by lowering the temperature of the cells to about 15° C., adding sodium-phosphate buffer to stabilize the pH at about 6.8 and centrifuging the culture medium to clarify it of cells). If the protein is secreted, the medium can be retained for further processing, if the protein is not secreted, the cells can be retained for further processing.

Any of several methods can be used to remove the cells from the medium, e.g., by centrifugation. For example, using a continuous disk-stack centrifuge, e.g., with a flow rate/sigma (cm/sec) of about 9.27×10⁻⁷.

Furthermore, the medium can be filtered to remove cells, e.g., by depth filtration with or without a centrifuge. For example, with a centrifuge, the process can, in an embodiment of the invention, comprise use of an 8±2 L broth/ft² filter (e.g., charged cellulose filter); without a centrifuge, the process can, in an embodiment of the invention, comprise use of a 20±3 L broth/ft² filter.

In addition, the medium can be filtered through a fine filter, for example, with a 0.2 micron pore size (e.g., a PVDF filter).

and;
4—Optionally further purifying the protein, e.g., antibody, for example, chromatographically.

In an embodiment of the invention, the enhanced process for producing a protein, such as an antibody (e.g., anti-IGF1 R) comprises the steps:

1—expanding cells expressing the protein in a standard initial mammalian growth media.

This expansion can be done, for example, in shake flasks. In an embodiment of the invention, expansion occurs by growth to about 1-2×10⁶ cells/ml, dilution of a sample of those cells (e.g., to a density of about 2.5-5×10⁵ cells/ml) and, then, regrowth to about 1-2×10⁶ cells/ml, for about 10-30 cycles.

2—inoculating an initial mammalian cell growth medium with the expanded cells, to a cell density of about 2.5-5×10⁵ cells/ml, and adding supplements to the medium. The supplements are soy and/or wheat hydrolysate, amino acid feed, vitamin/salt feed, nutrient feed, glucose and L-glutamine.

The day of inoculation is “day 0”, the following day is “day 1”, the following day is “day 2”, and so on.

Soy and/or wheat hydrolysates are added, for example, either on day 0 or after viable cell density has reached over about 10⁶ cells/ml. In an embodiment of the invention, the hydrolysate(s) are simply added on day 3.

Amino acid feeds (discussed above) are added, for example, at day 0, e.g., to reach approximate final culture concentrations as set forth below (not including the concentration of any indicated component from other sources such as from the initial mammalian cell growth medium):

| | Final concentration in |
|---|---|
| Component | culture (mg/liter) |
| L-arginine | 126.4 |
| L-cystine | 34 |
| L-histidine | 42 |
| L-isoleucine | 52 |
| L-leucine | 52 |
| L-lysine | 72 |
| L-Methionine | 15.2 |
| L-phenylalanine | 33 |
| L-threonine | 47.6 |
| L-tryptophan | 10.2 |
| L-tyrosine | 36 |
| L-valine | 46.8 |
| L-alanine | 8.9 |
| L-asparagine | 30 |
| L-aspartic acid | 26.6 |
| L-glutamic acid | 29.4 |
| Glycine | 15 |
| L-proline | 23 |
| L-serine | 21 |

In an embodiment of the invention, amino acid feeds are not added to the medium. Vitamin/salt feed solution (discussed above) is added, for example, between days 3 and 5 or when viable cell density reaches about 1.2×10⁶ cells/ml, e.g., to reach approximate final culture concentrations set forth below (not including the concentration of any indicated component from other sources such as from the initial mammalian cell growth medium):

| | Final culture concentrations |
|---|---|
| Component | (mg/liter) |
| Sodium selenite | 0.01426 |
| Adenine sulfate | 1.632 |
| Adenosine | 17.6 |
| Cytidine | 17.6 |
| Guanosine | 17.6 |
| Uridine | 17.6 |
| Hypoxanthine | 11.8 |
| L-citrulline | 12.6 |
| L-ornithine-HCl | 25.6 |
| Biotin | 0.28 |
| Flavin Adenine Dinucleotide | 0.05 |
| Folic Acid | 4.6 |
| Lipoic Acid | 0.52 |
| Niacin | 31.4 |
| Pyridoxine HCl | 3 |
| Riboflavin | 1.86 |
| Thiamine HCl | 16 |
| Vitamin E | 0.376 |
| Vitamin B12 | 3.4 |
| Choline Chloride | 50.2 |
| Ethanolamine HCl | 4.4 |
| i-Inositol | 73.2 |
| Thymidine | 7.8 |
| Putrescine 2HCl | 0.4 |
| Progesterone | 0.015 |
| D-Calcium Pantothenate | 23.8 |

Some components of the vitamin/salt feed are also in other feeds such as the amino acid feed. These final culture concentrations are of the components from the vitamin/salt feed and do not reflect the cumulative concentrations of the indicated components from both the amino acid feed and the vitamin/salt feed.

Nutrient feed (discussed above) is added, for example, between days 3 and 5 or when viable cell density reaches about 1.2×10⁶ cells/ml, e.g., to reach approximate final culture concentrations set forth below (not including the concentration of any indicated component from other sources such as from the initial mammalian cell growth medium or amino acid feed):

| | Final culture concentration | |
|---|---|---|
| Component | (mg/liter) | |
| L-asparagine: | 812 | mg/liter |
| L-proline | 216 | mg/liter |
| L-isoleucine | 370 | mg/liter |
| L-cysteine-HCl | 224 | mg/liter |
| L-leucine | 332 | mg/liter |
| L-threonine | 164 | mg/liter |
| L-tyrosine | 198 | mg/liter |
| L-arginine | 186 | mg/liter |
| L-aspartic acid | 71 | mg/liter |
| L-glutamic acid | 126 | mg/liter |
| Glycine | 57 | mg/liter |
| L-histidine | 125 | mg/liter |
| L-methionine | 132 | mg/liter |
| L-tryptophan | 99 | mg/liter |
| L-lysine | 293 | mg/liter |
| L-phenylalanine | 174 | mg/liter |
| L-valine | 262 | mg/liter |
| L-serine: | 260 | mg/liter |
| Sodium phosphate monobasic: | 288.2 | mg/liter |
| Zinc sulfate: | 1.08 | mg/liter |
| Cupric sulfate: | 0.0032 | mg/liter |
| Ammonium vanadate: | 0.00078 | mg/liter |
| Cobalt chloride: | 0.0025 | mg/liter |
| Nickel dichloride hexahydrate: | 0.0004 | mg/liter |
| Sodium molybdate dehydrate: | 0.00016 | mg/liter |
| Tin chloride dehydrate: | 0.00008 | mg/liter |
| Manganese chloride tetrahydrate: | 0.0003 | mg/liter |

Some components of the nutrient feed are also in other feeds such as the amino acid feed. These final culture concentrations are of the components from the nutrient feed and do not reflect the cumulative concentrations of the indicated components from both the amino acid feed and the nutrient feed.

Glucose is added, for example, when the glucose concentration in the culture medium falls below about 1.5 g/liter and L-glutamine is added, for example, when the glutamine concentration in the culture medium falls below about 150 mg/liter.

3—Optionally, harvesting the cells from the production cell culture medium, e.g., when viability is below 60%, by removing the cells from the culture medium (e.g., by lowering the temperature of the cells to about 15° C., adding sodium-phosphate buffer to stabilize the pH at about 6.8 and centrifuging the culture medium to clarify it of cells). If the protein is secreted, the medium can be retained for further processing, if the protein is not secreted, the cells can be retained for further processing.

Any of several methods can be used to remove the cells from the medium, e.g., by centrifugation. For example, using a continuous disk-stack centrifuge, e.g., with a flow rate/sigma (cm/sec) of about 9.27×10⁻⁷.

Furthermore, the medium can be filtered to remove cells, e.g., by depth filtration with or without a centrifuge. For example, with a centrifuge, the process can, in an embodiment of the invention, comprise use of an 8±2 L broth/ft² filter (e.g., charged cellulose filter); without a centrifuge, the process can, in an embodiment of the invention, comprise use of a 20±3 L broth/ft² filter.

In addition, the medium can be filtered through a fine filter, for example, with a 0.2 micron pore size (e.g., a PVDF filter).

and;
4—Optionally further purifying the protein, e.g., antibody, for example, chromatographically.

Optionally, when using either the level 3 or enhanced process, the osmolality of the culture is shifted to about 400 mOsm to about 500 mOsm (discussed above). In an embodiment of the invention, this shift occurs when the cells are at a density of at or above 1×10⁶ cells/ml.

Optionally, when using either the level 3 or enhanced process, the temperature of the culture is shifted to 33° C. to about 35° C. (discussed above). In an embodiment of the invention, this shift occurs, in an embodiment of the invention, between days 4 and 8, e.g., when the change in viable cell density over a 24 hour period is less than 10%.

In an embodiment of the invention, when using either the level 3 or enhanced process, the cell culture O₂ concentration, pH and temperature conditions are continuously monitored and adjusted during cell growth. In an embodiment of the invention O₂ concentration is monitored and maintained at about 60% during cell growth; and/or pH is continuously monitored and maintained at about 6.8 (e.g., ±0.02) during cell growth; and/or temperature is continuously monitored and maintained at about 36.5° C. (e.g., about ±0.5° C.) during cell growth.

Cell growth can be performed in any of several systems. For example, cell growth can be done in a simple flask, e.g., a glass shake flask. Other systems include tank bioreactors, bag bioreactors and disposable bioreactors. A tank bioreactor includes, typically, a metal vessel (e.g., a stainless steel jacketed vessel) in which cells are growth in a liquid medium. Tank bioreactors can be used for a wide range of culture volumes (e.g., 100 l, 150 l, 10000 l, 15000 l). Tank bioreactors often have additional features for controlling cell growth conditions, including means for temperature control, medium agitation, controlling sparge gas concentrations, controlling pH, controlling O₂ concentration, removing samples from the medium, reactor weight indication and control, cleaning hardware, sterilizing the hardware, piping or tubing to deliver all services, adding media, control pH, control solutions, and control gases, pumping sterile fluids into the growth vessel and, supervisory control and a data acquisition. Classifications of tank bioreactor include stirred tank reactors wherein mechanical stirrers (e.g., impellers) are used to mix the reactor to distribute heat and materials (such as oxygen and substrates). Bubble column reactors are tall reactors which use air alone to mix the contents. Air lift reactors are similar to bubble column reactors, but differ by the fact that they contain a draft tube. The draft tube is typically an inner tube which improves circulation and oxygen transfer and equalizes shear forces in the reactor. In fluidized bed reactors, cells are “immobilized” on small particles which move with the fluid. The small particles create a large surface area for cells to stick to and enable a high rate of transfer of oxygen and nutrients to the cells. In packed bed reactors cells are immobilized on large particles. These particles do not move with the liquid. Packed bed reactors are simple to construct and operate but can suffer from blockages and from poor oxygen transfer. A disposable bioreactor is a disposable, one-time use bioreactor. Often, disposable bioreactors possess features similar to non-disposable bioreactors (e.g., agitation system, sparge, probes, ports, etc.).

The present invention further includes any liquid culture medium generated by any of the processes set forth herein; for example, produced by a process comprising inoculating an initial mammalian cell growth medium, pre-warmed to about 37° C.; which medium comprises HEPES, sodium bicarbonate buffers, inorganic salts, non-essential amino acids, recombinant human insulin, trace elements and surfactants; and which does not comprise L-glutamine, antibiotics, antimycotics or animal-derived components; with CHO DXB11 host cells expressing the antibody light chain immunoglobulin and heavy chain immunoglobulin, to a cell density of about 2.5-5×10⁵ cells/ml; and, adding the following supplements to the medium before, simultaneously with or immediately after said inoculation: soy hydrolysate to a final concentration of about 10 g/liter;

and, optionally, an amino acid feed wherein the concentration of the components added by said amino acid feed are approximately those set forth below:

| | | |
|---|---|---|
| L-arginine: | 126.4 | mg/liter |
| L-cystine: | 34 | mg/liter |
| L-histidine: | 42 | mg/liter |
| L-isoleucine: | 52 | mg/liter |
| L-leucine: | 52 | mg/liter |
| L-lysine: | 72 | mg/liter |
| L-Methionine: | 15.2 | mg/liter |
| L-phenylalanine: | 33 | mg/liter |
| L-threonine: | 47.6 | mg/liter |
| L-tryptophan: | 10.2 | mg/liter |
| L-tyrosine: | 36 | mg/liter |
| L-valine: | 46.8 | mg/liter |
| L-alanine: | 8.9 | mg/liter |
| L-asparagine: | 30 | mg/liter |
| L-aspartic acid: | 26.6 | mg/liter |
| L-glutamic acid: | 29.4 | mg/liter |
| glycine: | 15 | mg/liter |
| L-proline: | 23 | mg/liter |
| L-serine: | 21 | mg/liter; |

and, when viable cell density reaches over about 1.2×10⁶ cells/ml, adding supplement feeds wherein the concentration of the components added by said supplement feeds are approximately those set forth below:

| | | |
|---|---|---|
| Sodium selenite: | 0.01426 | mg/liter |
| Adenine sulfate: | 1.632 | mg/liter |
| Adenosine: | 17.6 | mg/liter |
| Cytidine: | 17.6 | mg/liter |
| Guanosine: | 17.6 | mg/liter |
| Uridine: | 17.6 | mg/liter |
| Hypoxanthine: | 11.8 | mg/liter |
| L-citrulline: | 12.6 | mg/liter |
| L-ornithine-HCl: | 25.6 | mg/liter |
| Biotin: | 0.28 | mg/liter |
| Flavin Adenine Dinucleotide: | 0.05 | mg/liter |
| Folic Acid: | 4.6 | mg/liter |
| Lipoic Acid: | 0.52 | mg/liter |
| Niacin: | 31.4 | mg/liter |
| Pyridoxine HCl: | 3 | mg/liter |
| Riboflavin: | 1.86 | mg/liter |
| Thiamine HCl: | 16 | mg/liter |
| Vitamin E: | 0.376 | mg/liter |
| Vitamin B12: | 3.4 | mg/liter |
| Choline Chloride: | 50.2 | mg/liter |
| Ethanolamine HCl: | 4.4 | mg/liter |
| i-Inositol: | 73.2 | mg/liter |
| Thymidine: | 7.8 | mg/liter |
| Putrescine 2HCl: | 0.4 | mg/liter |
| Progesterone: | 0.015 | mg/liter |
| D-Calcium Pantothenate: | 23.8 | mg/liter |
| L-asparagine: | 812 | mg/liter |
| L-proline | 216 | mg/liter |
| L-isoleucine | 370 | mg/liter |
| L-cysteine-HCl | 224 | mg/liter |
| L-leucine | 332 | mg/liter |
| L-threonine | 164 | mg/liter |
| L-tyrosine | 198 | mg/liter |
| L-arginine | 186 | mg/liter |
| L-aspartic acid | 71 | mg/liter |
| L-glutamic acid | 126 | mg/liter |
| Glycine | 57 | mg/liter |
| L-histidine | 125 | mg/liter |
| L-methionine | 132 | mg/liter |
| L-tryptophan | 99 | mg/liter |
| L-lysine | 293 | mg/liter |
| L-phenylalanine | 174 | mg/liter |
| L-valine | 262 | mg/liter |
| L-serine: | 260 | mg/liter |
| Sodium phosphate monobasic: | 288.2 | mg/liter |
| Zinc sulfate: | 1.08 | mg/liter |
| Cupric sulfate: | 0.0032 | mg/liter |
| Ammonium vanadate: | 0.00078 | mg/liter |
| Cobalt chloride: | 0.0025 | mg/liter |
| Nickel dichloride hexahydrate: | 0.0004 | mg/liter |
| Sodium molybdate dehydrate: | 0.00016 | mg/liter; |

and, during cell growth, adding glucose to the medium when glucose levels fall below about 1.5 g/liter and adding L-glutamine when L-glutamine levels fall below about 150 mg/liter; and during cell growth maintaining O₂ concentration at about 60%; pH at about 6.8±0.02 and temperature at about 36.5° C.±0.5° C.; for example, wherein the medium comprises cells that have reached 60% viability.

### EXAMPLES

The following information is provided for more clearly describing the present invention and should not be construed to limit the present invention. Any and all of the compositions and methods described below, in whole or in part, fall within the scope of the present invention.

### Example 1

### Expression of Anti-IGF1 R Using Level 3 and Enhanced Process

Several runs using the enhanced process and the level 3 process were performed. In these runs, CHO DXB11 cells expressing the anti-IGF1R LCF (kappa) and HCA (gamma-1) chains were grown. The initial mammalian cell growth medium to which supplements were added was the EX-CELL ACF CHO medium (Sigma-Aldrich; St. Louis, Mo.).

A similar set of runs were performed wherein there were no additions of feeds (except for in-process glutamine and glucose). Under these conditions, a titer of 435 mg/L was obtained. This titer was estimated by quantitating the immunoglobulin produced which adhered specifically to protein A. The titers obtained in the following level 3 and enhanced process runs were estimated from quantitating immunoglobulin that adhered to a reverse phase chromatography substrate. An estimated titer of about 300 mg/L would have been obtained in the run without addition of feeds had the reverse phase method of quantitation been used.

Relative to the titer obtained in the run excluding the addition of feeds, the level 3 and enhanced processes (discussed below) produce far superior titers.

Enhanced Process

Cells were initially inoculated at 3-4×10⁵ cells/ml in the EX-CELL ACF CHO medium (Sigma-Aldrich; St. Louis, Mo.) which was pre-warmed to 37° C. and adjusted to pH 6.8.

TABLE 1
Feeds added for enhanced process runs.

| | | | Volume | |
|---|---|---|---|---|
| | | | ratio | |
| | | | (volume | In-process |
| | | Time | feed/volume | temperature |
| Batch | Feeds added | added | batch) | downshift |
| 1 | SHYS feed | Day 0 | 0.05 | Yes: day 6 |
| | CHO feed 1 | Day 3 | 0.02 | from 37° C. to |
| | CHO feed 2 | Day 3 | 0.02 | 34° C. |
| 2 | SHYS feed | Day 0 | 0.05 | Yes: day 6 |
| | CHO feed 1 | Day 3 | 0.02 | from 37° C. to |
| | CHO feed 2 | Day 3 | 0.02 | 34° C. |
| 3 | HYS feed | Day 0 | 0.05 | Yes: day 6 |
| | CHO feed 1 | Day 3 | 0.02 | from 37° C. to |
| | CHO feed 2 | Day 3 | 0.02 | 34° C. |
| 4 | HYS feed | Day 0 | 0.05 | Yes: day 6 |
| | CHO feed 1 | Day 3 | 0.02 | from 37° C. to |
| | CHO feed 2 | Day 3 | 0.02 | 34° C. |
| 5 | SHYS feed | Day 0 | 0.05 | No |
| | 50X amino acid feed | Day 0 | 0.02 | |
| | 100X amino acid | Day 0 | 0.01 | |
| | feed | | | |
| | CHO feed 1 | Day 3 | 0.02 | |
| | CHO feed 2 | Day 3 | 0.02 | |

SHYS feed: a 200 g/L (aq) soy hydrolysate feed from DMV international (Netherlands)
HYS feed: a 200 g/L (aq) soy hydrolysate feed from Kerry Biosciences
CHO feed 1: 50X Vitamin/salt feed
CHO feed 2: 50X Nutrient feed
50X amino acid feed
100X amino acid feed
pH was continuously monitored and adjusted to a setpoint of 6.8.
Oxygen concentration was continuously monitored and adjusted to a setpoint of 60%.
Temperature was continuously monitored and maintained at 37 ± 1° C. An in-process temperature downshift to 34° C. was performed in the indicated batches.

Glucose was added, for example, when the glucose concentration in the culture medium fell below 1.5 g/liter and L-glutamine was added, for example, when the glutamine concentration in the culture medium fell below 150 mg/liter. The osmolality was shifted to over 400 mOsm from addition of the nutrient feed. The cells were harvested between days 21-24, except for batches 3 and 4 which were harvested earlier (days 14-18); generally, when cell viability was reduced to about 60%.

TABLE 2
Results of enhanced process runs.

| Batch | Titer | Specific productivity |
|---|---|---|
| 1 | 2.2 | 26 |
| 2 | 2.3 | 27 |
| 3* | 1.9 | 30 |
| 4* | 1.6 | 33 |
| 5 | 2.2 | 26 |

*Abbreviated batch run

The addition of amino acid feeds may be omitted when the nutrient feeds are used in the enhanced process.

### Level 3 Process

Cells were initially inoculated at 3-4×10⁵ cells/ml in the EX-CELL ACF CHO medium (Sigma-Aldrich; St. Louis, Mo.) which was pre-warmed to 37° C. and adjusted to pH 6.8.

TABLE 3
Feeds added for level 3 process

| | | | Volume ratio | |
|---|---|---|---|---|
| | | | (volume | In-process |
| | | Time | feed/volume | temperature |
| Batch | Feeds added | added | batch) | downshift |
| A | SHYS feed | Day 0 | 0.05 | No |
| | 50X amino acid feed | Day 0 | 0.02 | |
| | 100X amino acid | Day 0 | 0.01 | |
| | feed | | | |
| | CHO feed 1 | Day 3 | 0.02 | |
| B | SHYS feed | Day 0 | 0.05 | No |
| | 50X amino acid feed | Day 0 | 0.02 | |
| | 100X amino acid | Day 0 | 0.01 | |
| | feed | | | |
| | CHO feed 1 | Day 3 | 0.02 | |

SHYS feed: a 200 g/L (aq) soy hydrolysate feed from DMV international (Netherlands)
CHO feed 1: 50X Vitamin/salt feed
50X amino acid feed
100X amino acid feed
pH was continuously monitored and maintained at 6.8 ± 0.02.
Oxygen concentration was continuously monitored and adjusted to a setpoint of 60%.
Temperature was continuously monitored and maintained at 36.5 ± 0.5° C.

Glucose was added, for example, when the glucose concentration in the culture medium fell below 1.5 g/liter and L-glutamine was added, for example, when the glutamine concentration in the culture medium fell below 150 mg/liter.

TABLE 4
Results of level 3 process runs.

| | Titer | Specific |
|---|---|---|
| Batch | (g/L) | productivity |
| A | 1.4 | 19 |
| B | 1.2 | 22 |

The feeds used and the final concentrations of the components of each feed are set forth below.

TABLE 5
Vitamin/salt feed

| | Concentration in | Final concentration in |
|---|---|---|
| Component | feed (g/L) | culture (mg/L) |
| Sodium selenite | 7.13 × 10⁻⁴ | 0.01426 |
| Adenine sulfate | 0.0816 | 1.632 |
| Adenosine | 0.88 | 17.6 |
| Cytidine | 0.88 | 17.6 |
| Guanosine | 0.88 | 17.6 |
| Uridine | 0.88 | 17.6 |
| Hypoxanthine | 0.59 | 11.8 |
| L-citrulline | 0.63 | 12.6 |
| L-ornithine-HCl | 1.28 | 25.6 |
| Biotin | 0.014 | 0.28 |
| Flavin Adenine Dinucleotide | 0.0025 | 0.05 |
| Folic Acid | 0.23 | 4.6 |
| Lipoic Acid | 0.026 | 0.52 |
| Niacin | 1.57 | 31.4 |
| Pyridoxine HCl | 0.15 | 3 |
| Riboflavin | 0.093 | 1.86 |
| Thiamine HCl | 0.8 | 16 |
| Vitamin E | 0.0188 | 0.376 |
| Vitamin B12 | 0.17 | 3.4 |
| Choline Chloride | 2.51 | 50.2 |
| Ethanolamine HCl | 0.22 | 4.4 |
| i-Inositol | 3.66 | 73.2 |
| Thymidine | 0.39 | 7.8 |
| Putrescine 2HCl | 0.02 | 0.4 |
| Progesterone | 0.00075 | 0.015 |
| D-Calcium Pantothenate | 1.19 | 23.8 |

TABLE 6
Amino acid feed #1 (50X).

| | Concentration | Final concentration in culture |
|---|---|---|
| Component | in feed (g/L) | (mg/L) |
| L-arginine | 6.32 | 126.4 |
| L-cystine | 1.7 | 34 |
| L-histidine | 2.1 | 42 |
| L-isoleucine | 2.6 | 52 |
| L-leucine | 2.6 | 52 |
| L-lysine | 3.6 | 72 |
| L-Methionine | 0.76 | 15.2 |
| L-phenylalanine | 1.65 | 33 |
| L-threonine | 2.38 | 47.6 |
| L-tryptophan | 0.51 | 10.2 |
| L-tyrosine | 1.8 | 36 |
| L-valine | 2.34 | 46.8 |

TABLE 7
Amino acid feed #2 (100X).

| | Concentration | Final concentration in culture |
|---|---|---|
| Component | in feed (g/L) | (mg/L) |
| L-alanine | 0.89 | 8.9 |
| L-asparagine | 1.5 | 30 |
| L-aspartic acid | 1.33 | 26.6 |
| L-glutamic acid | 1.47 | 29.4 |
| Glycine | 0.75 | 15 |
| L-proline | 1.15 | 23 |
| L-serine | 1.05 | 21 |

TABLE 8
Nutrient feed.

| | Concentration | Final concentration in |
|---|---|---|
| Component | in feed (g/L) | culture (mg/L) |
| L-asparagine | 40.6 | 812 |
| L-serine | 13 | 260 |
| L-proline | 10.81 | 216 |
| L-isoleucine | 18.53 | 370 |
| L-cysteine-HCl | 11.19 | 224 |
| L-leucine | 16.58 | 332 |
| L-threonine | 8.2 | 164 |
| L-tyrosine | 9.9 | 198 |
| L-arginine | 9.29 | 186 |
| L-aspartic acid | 3.56 | 71 |
| L-glutamic acid | 6.28 | 126 |
| glycine | 2.83 | 57 |
| L-histidine | 6.23 | 125 |
| L-methionine | 6.58 | 132 |
| L-tryptophan | 4.93 | 99 |
| L-lysine | 14.66 | 293 |
| L-phenylalanine | 8.64 | 174 |
| L-valine | 13.08 | 262 |
| Sodium phosphate monobasic | 14.41 | 288.2 |
| Zinc sulfate | 0.054 | 1.08 |
| Cupric sulfate | 0.00016 | 0.0032 |
| Ammonium vanadate | 0.000039 | 0.00078 |
| Ccobalt chloride | 0.000125 | 0.0025 |
| Nickel dichloride hexahydrate | 0.00002 | 0.0004 |
| Sodium molybdate dihydrate | 0.000008 | 0.00016 |
| Tin chloride dihydrate | 0.000004 | 0.00008 |
| Manganese chloride tetrahydrate | 0.000015 | 0.0003 |

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

We claim:

1. A method for producing a protein comprising inoculating an initial mammalian cell growth medium with host cells expressing the protein and adding supplements comprising:
Glucose;
L-glutamine;
Soy hydrolysate or wheat hydrolysate or both;
Adenine sulfate;
Adenosine;
ammonium vanadate;
Biotin;
Choline Chloride;
Cobalt chloride;
Cupric sulfate;
Cytidine;
D-Calcium Pantothenate;
Ethanolamine HCl;
Flavin Adenine Dinucleotide;
Folic Acid;
Glycine;
Guanosine;
Hypoxanthine;
i-Inositol;
L-alanine;
L-arginine;
L-asparagine;
L-aspartic acid;
L-citrulline;
L-cysteine-HCl;
L-cystine;
L-glutamic acid;
L-histidine;
Lipoic Acid;
L-isoleucine;
L-leucine;
L-lysine;
L-methionine;
L-ornithine-HCl;
L-phenylalanine;
L-proline;
L-serine;
L-threonine;
L-tryptophan;
L-tyrosine;
L-valine;
Manganese chloride Tetrahydrate;
Niacin;
Nickel dichloride hexahydrate;
Progesterone;
Putrescine 2HCl;
Pyridoxine HCl;
Riboflavin;
Sodium molybdate dehydrate;
Sodium phosphate monobasic;
Sodium selenite;
Thiamine HCl;
Thymidine;
Tin chloride dehydrate;
Uridine;
Vitamin B12;
Vitamin E; and
Zinc sulfate;
to the medium.

2. The method of claim 1 wherein the final concentration of the components added to the medium from the supplements are about those set forth below:

| | | |
|---|---|---|
| Adenine sulfate: | 1.632 | mg/liter |
| Adenosine: | 17.6 | mg/liter |
| Ammonium vanadate: | 0.00078 | mg/liter |
| Biotin: | 0.28 | mg/liter |
| Choline Chloride: | 50.2 | mg/liter |
| Cobalt chloride: | 0.0025 | mg/liter |
| Cupric sulfate: | 0.0032 | mg/liter |
| Cytidine: | 17.6 | mg/liter |
| D-Calcium Pantothenate: | 23.8 | mg/liter |
| Ethanolamine HCl: | 4.4 | mg/liter |
| Flavin Adenine Dinucleotide: | 0.05 | mg/liter |
| Folic Acid: | 4.6 | mg/liter |
| Glycine | 72 | mg/liter |
| Guanosine: | 17.6 | mg/liter |
| Hypoxanthine: | 11.8 | mg/liter |
| i-Inositol: | 73.2 | mg/liter |
| L-alanine: | 8.9 | mg/liter |
| L-arginine | 312.4 | mg/liter |
| L-asparagine: | 842 | mg/liter |
| L-aspartic acid | 97.6 | mg/liter |
| L-citrulline: | 12.6 | mg/liter |
| L-cysteine-HCl | 224 | mg/liter |
| L-cystine: | 34 | mg/liter |
| L-glutamic acid | 155.4 | mg/liter |
| L-histidine | 167 | mg/liter |
| Lipoic Acid: | 0.52 | mg/liter |
| L-isoleucine | 422 | mg/liter |
| L-leucine | 384 | mg/liter |
| L-lysine | 365 | mg/liter |
| L-methionine | 147.2 | mg/liter |
| L-ornithine-HCl: | 25.6 | mg/liter |
| L-phenylalanine | 207 | mg/liter |
| L-proline | 239 | mg/liter |
| L-serine: | 281 | mg/liter |
| L-threonine | 211.6 | mg/liter |
| L-tryptophan | 109.2 | mg/liter |
| L-tyrosine | 234 | mg/liter |
| L-valine | 308.8 | mg/liter |
| Manganese chloride tetrahydrate: | 0.0003 | mg/liter |
| Niacin: | 31.4 | mg/liter |
| Nickel dichloride hexahydrate: | 0.0004 | mg/liter |
| Progesterone: | 0.015 | mg/liter |
| Putrescine 2HCl: | 0.4 | mg/liter |
| Pyridoxine HCl: | 3 | mg/liter |
| Riboflavin: | 1.86 | mg/liter |
| Sodium molybdate dehydrate: | 0.00016 | mg/liter |
| Sodium phosphate monobasic: | 288.2 | mg/liter |
| Sodium selenite: | 0.01426 | mg/liter |
| Thiamine HCl: | 16 | mg/liter |
| Thymidine: | 7.8 | mg/liter |
| Tin chloride dehydrate: | 0.00008 | mg/liter |
| Uridine: | 17.6 | mg/liter |
| Vitamin B12: | 3.4 | mg/liter |
| Vitamin E: | 0.376 | mg/liter |
| Zinc sulfate: | 1.08 | mg/liter |
| Glucose | 1.5 | g/liter |
| L-glutamine | 150 | mg/liter |

3. The method of claim 1 wherein the supplements are added from an amino acid feed that comprises amino acids at about the following concentrations:

| | | |
|---|---|---|
| L-arginine: | 6.32 | g/liter |
| L-cystine: | 1.7 | g/liter |
| L-histidine: | 2.1 | g/liter |
| L-isoleucine: | 2.6 | g/liter |
| L-leucine: | 2.6 | g/liter |
| L-lysine: | 3.6 | g/liter |
| L-Methionine: | 0.76 | g/liter |
| L-phenylalanine: | 1.65 | g/liter |
| L-threonine: | 2.38 | g/liter |
| L-tryptophan: | 0.51 | g/liter |
| L-tyrosine: | 1.8 | g/liter |
| L-valine: | 2.34 | g/liter |

4. The method of claim 3 wherein about 20 ml amino acid feed is added per liter of culture medium.

5. The method of claim 1 wherein the supplements are added from an amino acid feed that comprises amino acids at about the following concentrations:

| | | |
|---|---|---|
| L-alanine: | 0.89 | g/liter |
| L-asparagine: | 1.5 | g/liter |
| L-aspartic acid: | 1.33 | g/liter |
| L-glutamic acid: | 1.47 | g/liter |
| Glycine: | 0.75 | g/liter |
| L-proline: | 1.15 | g/liter |
| L-serine: | 1.05 | g/liter |

6. The method of claim 5 wherein about 10 ml amino acid feed is added per liter of culture medium.

7. The method of claim 1 wherein the supplements are added from a nutrient feed that comprises supplements at about the following concentrations:

| | | |
|---|---|---|
| L-asparagine: | 40.6 | g/liter |
| L-proline | 10.81 | g/liter |
| L-isoleucine | 18.53 | g/liter |
| L-cysteine-HCl | 11.19 | g/liter |
| L-leucine | 16.58 | g/liter |
| L-threonine | 8.2 | g/liter |
| L-tyrosine | 9.9 | g/liter |
| L-arginine | 9.29 | g/liter |
| L-aspartic acid | 3.56 | g/liter |
| L-glutamic acid | 6.28 | g/liter |
| Glycine | 2.83 | g/liter |
| L-histidine | 6.23 | g/liter |
| L-methionine | 6.58 | g/liter |
| L-tryptophan | 4.93 | g/liter |
| L-lysine | 14.66 | g/liter |
| L-phenylalanine | 8.64 | g/liter |
| L-valine | 13.08 | g/liter |
| L-serine: | 13 | g/liter |
| Sodium phosphate Monobasic: | 14.41 | g/liter |
| Zinc sulfate: | 0.054 | g/liter |
| Cupric sulfate: | 0.00016 | g/liter |
| Ammonium vanadate: | 0.000039 | g/liter |
| Cobalt chloride: | 0.000125 | g/liter |
| Nickel dichloride Hexahydrate: | 0.00002 | g/liter |
| Sodium molybdate dehydrate: | 0.000008 | g/liter |
| Tin chloride dehydrate: | 0.000004 | g/liter |
| Manganese chloride: tetrahydrate: | 0.000015 | g/liter. |

8. The method of claim 7 wherein about 20 ml nutrient feed is added per liter of culture medium.

9. The method of claim 1 wherein the supplements are added from a vitamin/salt feed that comprises supplements at about the following concentrations:

| Sodium selenite: | 7.13 × 10⁻⁴ | g/liter |
|---|---|---|
| Adenine sulfate: | 0.0816 | g/liter |
| Adenosine: | 0.88 | g/liter |
| Cytidine: | 0.88 | g/liter |
| Guanosine: | 0.88 | g/liter |
| Uridine: | 0.88 | g/liter |
| Hypoxanthine: | 0.59 | g/liter |
| L-citrulline: | 0.63 | g/liter |
| L-ornithine-HCl: | 1.28 | g/liter |
| Biotin: | 0.014 | g/liter |
| Flavin Adenine Dinucleotide: | 0.0025 | g/liter |
| Folic Acid: | 0.23 | g/liter |
| Lipoic Acid: | 0.026 | g/liter |
| Niacin: | 1.57 | g/liter |
| Pyridoxine HCl: | 0.15 | g/liter |
| Riboflavin: | 0.093 | g/liter |
| Thiamine HCl: | 0.8 | g/liter |
| Vitamin E: | 0.0188 | g/liter |
| Vitamin B12: | 0.17 | g/liter |
| Choline Chloride: | 2.51 | g/liter |
| Ethanolamine HCl: | 0.22 | g/liter |
| i-Inositol: | 3.66 | g/liter |
| Thymidine: | 0.39 | g/liter |
| Putrescine 2HCl: | 0.02 | g/liter |
| Progesterone: | 0.00075 | g/liter |
| D-Calcium Pantothenate: | 1.19 | g/liter |

10. The method of claim 9 wherein about 20 ml vitamin/salt feed is added per liter of culture medium.

11. The method of claim 1 further comprising harvesting the medium from the host cells; wherein said protein is secreted from said cells into the medium.

12. The method of claim 13 wherein the culture medium is harvested from the host cells when viability of the cells is below about 60%.

13. The method of claim 11 further comprising purifying the culture medium from the cells by centrifuging the medium and/or depth filtering the medium and/or filtering the medium through a 0.2 micron filter.

14. The method of claim 1 wherein the protein is one or more immunoglobulin chains of an antibody or antigen-binding fragment thereof.

15. The method of claim 14 wherein the antibody or fragment binds specifically to IGF1R.

16. The method of claim 15 wherein the antibody or fragment comprises a heavy immunoglobulin chain comprising the amino acid sequence set forth in a member selected from the group consisting of SEQ ID NOs: 1, 3,-6, 13, 21 and 26; or a mature fragment thereof or one or more CDRs therefrom; and/or
a light immunoglobulin chain comprising an amino acid sequence selected from the group consisting of SEQ ID NOs: 2, 7-9, 17 and 25, or a mature fragment thereof or one or more CDRs therefrom.

17. The method of claim 16 wherein the antibody or fragment secreted from the host cell is an antibody that comprises a light chain immunoglobulin comprising amino acids 20-128 of SEQ ID NO: 3, 4, 5, or 6; and a heavy chain immunoglobulin comprising amino acids 20-137 of SEQ ID NO: 8 or 9.

18. The method of claim 17 wherein the light chain immunoglobulin is linked to a kappa constant immunoglobulin chain and the heavy chain immunoglobulin is linked to a gamma-1 constant immunoglobulin chain.

19. The method of claim 1 wherein the initial mammalian cell growth medium to which the supplements are added comprises HEPES, sodium bicarbonate buffers, inorganic salts, non-essential amino acids, recombinant human insulin, trace elements and surfactants; and does not comprise L-glutamine, antibiotics, antimycotics or animal-derived components.

20. A method for producing an antibody comprising inoculating an initial mammalian cell growth medium, pre-warmed to about 37° C.; which initial medium comprises HEPES, sodium bicarbonate buffers, inorganic salts, non-essential amino acids, recombinant human insulin, trace elements and surfactants; and which does not comprise L-glutamine, antibiotics, antimycotics or animal-derived components; with CHO DXB11 host cells expressing the antibody light chain immunoglobulin and heavy chain immunoglobulin, to a cell density of about 2.5-5×10⁵ cells/ml; and, adding the following supplements to the medium before, simultaneously with or immediately after said inoculation: soy hydrolysate to a final concentration of about 10 g/liter; and, optionally, an amino acid feed wherein the concentration of the components added by said amino acid feed are approximately those set forth below:

| | | |
|---|---|---|
| L-arginine: | 126.4 | mg/liter |
| L-cystine: | 34 | mg/liter |
| L-histidine: | 42 | mg/liter |
| L-isoleucine: | 52 | mg/liter |
| L-leucine: | 52 | mg/liter |
| L-lysine: | 72 | mg/liter |
| L-Methionine: | 15.2 | mg/liter |
| L-phenylalanine: | 33 | mg/liter |
| L-threonine: | 47.6 | mg/liter |
| L-tryptophan: | 10.2 | mg/liter |
| L-tyrosine: | 36 | mg/liter |
| L-valine: | 46.8 | mg/liter |
| L-alanine: | 8.9 | mg/liter |
| L-asparagine: | 30 | mg/liter |
| L-aspartic acid: | 26.6 | mg/liter |
| L-glutamic acid: | 29.4 | mg/liter |
| glycine: | 15 | mg/liter |
| L-proline: | 23 | mg/liter |
| L-serine: | 21 | mg/liter; |

and, when viable cell density reaches over about 1.2×10⁶ cells/ml, adding supplement feeds wherein the concentration of the components added by said supplement feeds are approximately those set forth below:

| | | |
|---|---|---|
| Sodium selenite: | 0.01426 | mg/liter |
| Adenine sulfate: | 1.632 | mg/liter |
| Adenosine: | 17.6 | mg/liter |
| Cytidine: | 17.6 | mg/liter |
| Guanosine: | 17.6 | mg/liter |
| Uridine: | 17.6 | mg/liter |
| Hypoxanthine: | 11.8 | mg/liter |
| L-citrulline: | 12.6 | mg/liter |
| L-ornithine-HCl: | 25.6 | mg/liter |
| Biotin: | 0.28 | mg/liter |
| Flavin Adenine Dinucleotide: | 0.05 | mg/liter |
| Folic Acid: | 4.6 | mg/liter |
| Lipoic Acid: | 0.52 | mg/liter |
| Niacin: | 31.4 | mg/liter |
| Pyridoxine HCl: | 3 | mg/liter |
| Riboflavin: | 1.86 | mg/liter |
| Thiamine HCl: | 16 | mg/liter |
| Vitamin E: | 0.376 | mg/liter |
| Vitamin B12: | 3.4 | mg/liter |
| Choline Chloride: | 50.2 | mg/liter |
| Ethanolamine HCl: | 4.4 | mg/liter |
| i-Inositol: | 73.2 | mg/liter |
| Thymidine: | 7.8 | mg/liter |
| Putrescine 2HCl: | 0.4 | mg/liter |
| Progesterone: | 0.015 | mg/liter |
| D-Calcium Pantothenate: | 23.8 | mg/liter |
| L-asparagine: | 812 | mg/liter |
| L-proline | 216 | mg/liter |
| L-isoleucine | 370 | mg/liter |
| L-cysteine-HCl | 224 | mg/liter |
| L-leucine | 332 | mg/liter |
| L-threonine | 164 | mg/liter |
| L-tyrosine | 198 | mg/liter |
| L-arginine | 186 | mg/liter |
| L-aspartic acid | 71 | mg/liter |
| L-glutamic acid | 126 | mg/liter |
| Glycine | 57 | mg/liter |
| L-histidine | 125 | mg/liter |
| L-methionine | 132 | mg/liter |
| L-tryptophan | 99 | mg/liter |
| L-lysine | 293 | mg/liter |
| L-phenylalanine | 174 | mg/liter |
| L-valine | 262 | mg/liter |
| L-serine: | 260 | mg/liter |
| Sodium phosphate monobasic: | 288.2 | mg/liter |
| Zinc sulfate: | 1.08 | mg/liter |
| Cupric sulfate: | 0.0032 | mg/liter |
| Ammonium vanadate: | 0.00078 | mg/liter |
| Cobalt chloride: | 0.0025 | mg/liter |
| Nickel dichloride hexahydrate: | 0.0004 | mg/liter |
| Sodium molybdate dehydrate: | 0.00016 | mg/liter; |

and, maintaining glucose concentration in the medium at about 1.5 g/liter and maintaining L-glutamine concentration in the medium at about 150 mg/liter; and during cell growth maintaining O₂ concentration at about 60%; pH at about 6.8±0.02 and temperature at about 36.5° C.±0.5° C.; and, optionally, removing the host cells from the medium when cell viability is below about 60%.

21. The method of claim 20 wherein the antibody comprises a heavy immunoglobulin chain comprising the amino acid sequence set forth in a member selected from the group consisting of SEQ ID NOs: 1, 3,-6, 13, 21 and 26; or a mature fragment thereof or one or more CDRs therefrom; and/or
a light immunoglobulin chain comprising an amino acid sequence selected from the group consisting of SEQ ID NOs: 2, 7-9, 17 and 25, or a mature fragment thereof or one or more CDRs therefrom.

22. The method of claim 21 wherein the antibody or fragment comprises an antibody secreted from the host cells that comprises a light chain immunoglobulin comprising amino acids 20-128 of SEQ ID NO: 3, 4, 5, or 6; and a heavy chain immunoglobulin comprising amino acids 20-137 of SEQ ID NO: 8 or 9.

23. The method of claim 22 wherein the light chain immunoglobulin is linked to a kappa constant immunoglobulin chain and the heavy chain immunoglobulin is linked to a gamma-1 constant immunoglobulin chain.

24. The method of claim 23 further comprising recovering the culture medium from the cells by disk-stack centrifuging the medium, depth filtering the medium and filtering the medium through a filter with about a 0.2 micron pore size.

25. The method of claim 24 further comprising purifying the immunoglobulins from the medium by column chromatographic fractionation.

26. An aqueous liquid cell culture medium comprising:
about 10 g/liter soy hydrolysate,
about 1.5 g/liter glucose,
about 150 mg/liter L-glutamine,
pH of about 6.8±0.02,
HEPES,
Sodium bicarbonate buffers,
Inorganic salts,
Non-essential amino acids,
Recombinant human insulin,
Trace elements,
Surfactants,
an amino acid feed wherein the concentration of the components added by said amino acid feed are approximately those set forth below:

| | | |
|---|---|---|
| L-arginine: | 126.4 | mg/liter |
| L-cystine: | 34 | mg/liter |
| L-histidine: | 42 | mg/liter |
| L-isoleucine: | 52 | mg/liter |
| L-leucine: | 52 | mg/liter |
| L-lysine: | 72 | mg/liter |
| L-Methionine: | 15.2 | mg/liter |
| L-phenylalanine: | 33 | mg/liter |
| L-threonine: | 47.6 | mg/liter |
| L-tryptophan: | 10.2 | mg/liter |
| L-tyrosine: | 36 | mg/liter |
| L-valine: | 46.8 | mg/liter |
| L-alanine: | 8.9 | mg/liter |
| L-asparagine: | 30 | mg/liter |
| L-aspartic acid: | 26.6 | mg/liter |
| L-glutamic acid: | 29.4 | mg/liter |
| Glycine: | 15 | mg/liter |
| L-proline: | 23 | mg/liter |
| L-serine: | 21 | mg/liter; |

wherein the medium does not comprise antibiotics, antimycotics or animal-derived components.

27. An aqueous liquid culture medium produced by a process comprising inoculating an initial mammalian cell growth medium, pre-warmed to about 37° C.; which medium comprises HEPES, sodium bicarbonate buffers, inorganic salts, non-essential amino acids, recombinant human insulin, trace elements and surfactants; and which does not comprise L-glutamine, antibiotics, antimycotics or animal-derived components; with CHO DXB11 host cells expressing an antibody light chain immunoglobulin and heavy chain immunoglobulin, to a cell density of about 2.5-5×10⁵ cells/ml; and, adding the following supplements to the medium before, simultaneously with or immediately after said inoculation:
soy hydrolysate to a final concentration of about 10 g/liter;
and, optionally, an amino acid feed wherein the concentration of the components added by said amino acid feed are approximately those set forth below:

| | | |
|---|---|---|
| L-arginine: | 126.4 | mg/liter |
| L-cystine: | 34 | mg/liter |
| L-histidine: | 42 | mg/liter |
| L-isoleucine: | 52 | mg/liter |
| L-leucine: | 52 | mg/liter |
| L-lysine: | 72 | mg/liter |
| L-Methionine: | 15.2 | mg/liter |
| L-phenylalanine: | 33 | mg/liter |
| L-threonine: | 47.6 | mg/liter |
| L-tryptophan: | 10.2 | mg/liter |
| L-tyrosine: | 36 | mg/liter |
| L-valine: | 46.8 | mg/liter |
| L-alanine: | 8.9 | mg/liter |
| L-asparagine: | 30 | mg/liter |
| L-aspartic acid: | 26.6 | mg/liter |
| L-glutamic acid: | 29.4 | mg/liter |
| Glycine: | 15 | mg/liter |
| L-proline: | 23 | mg/liter |
| L-serine: | 21 | mg/liter; |

and, when viable cell density reaches over about 1.2×10⁶ cells/ml, adding supplement feeds wherein the concentration of the components added by said supplement feeds are approximately those set forth below:

| | | |
|---|---|---|
| Sodium selenite: | 0.01426 | mg/liter |
| Adenine sulfate: | 1.632 | mg/liter |
| Adenosine: | 17.6 | mg/liter |
| Cytidine: | 17.6 | mg/liter |
| Guanosine: | 17.6 | mg/liter |
| Uridine: | 17.6 | mg/liter |
| Hypoxanthine: | 11.8 | mg/liter |
| L-citrulline: | 12.6 | mg/liter |
| L-ornithine-HCl: | 25.6 | mg/liter |
| Biotin: | 0.28 | mg/liter |
| Flavin Adenine Dinucleotide: | 0.05 | mg/liter |
| Folic Acid: | 4.6 | mg/liter |
| Lipoic Acid: | 0.52 | mg/liter |
| Niacin: | 31.4 | mg/liter |
| Pyridoxine HCl: | 3 | mg/liter |
| Riboflavin: | 1.86 | mg/liter |
| Thiamine HCl: | 16 | mg/liter |
| Vitamin E: | 0.376 | mg/liter |
| Vitamin B12: | 3.4 | mg/liter |
| Choline Chloride: | 50.2 | mg/liter |
| Ethanolamine HCl: | 4.4 | mg/liter |
| i-Inositol: | 73.2 | mg/liter |
| Thymidine: | 7.8 | mg/liter |
| Putrescine 2HCl: | 0.4 | mg/liter |
| Progesterone: | 0.015 | mg/liter |
| D-Calcium Pantothenate: | 23.8 | mg/liter |
| L-asparagine: | 812 | mg/liter |
| L-proline | 216 | mg/liter |
| L-isoleucine | 370 | mg/liter |
| L-cysteine-HCl | 224 | mg/liter |
| L-leucine | 332 | mg/liter |
| L-threonine | 164 | mg/liter |
| L-tyrosine | 198 | mg/liter |
| L-arginine | 186 | mg/liter |
| L-aspartic acid | 71 | mg/liter |
| L-glutamic acid | 126 | mg/liter |
| Glycine | 57 | mg/liter |
| L-histidine | 125 | mg/liter |
| L-methionine | 132 | mg/liter |
| L-tryptophan | 99 | mg/liter |
| L-lysine | 293 | mg/liter |
| L-phenylalanine | 174 | mg/liter |
| L-valine | 262 | mg/liter |
| L-serine: | 260 | mg/liter |
| Sodium phosphate monobasic: | 288.2 | mg/liter |
| Zinc sulfate: | 1.08 | mg/liter |
| Cupric sulfate: | 0.0032 | mg/liter |
| Ammonium vanadate: | 0.00078 | mg/liter |
| Cobalt chloride: | 0.0025 | mg/liter |
| Nickel dichloride hexahydrate: | 0.0004 | mg/liter |
| Sodium molybdate dehydrate: | 0.00016 | mg/liter; |

and, maintaining glucose concentration in the medium at about 1.5 g/liter and maintaining L-glutamine concentration in the medium at about 150 mg/liter; and during cell growth maintaining O₂ concentration at about 60%; pH at about 6.8±0.02 and temperature at about 36.5° C.±0.5° C.

28. The aqueous liquid culture medium of claim 27 wherein the host cells comprise a vector encoding the immunoglobulins of the antibody wherein the antibody is secreted into the medium.

29. The aqueous liquid culture medium of claim 28 wherein the host cell viability is about 60% or lower and/or wherein host cell growth has proceeded for about 14 to 24 days.

30. The aqueous liquid culture medium of claim 29 wherein the host cells are removed from the medium.

31. The aqueous liquid culture medium of claim 30 wherein the host cells are removed from the medium by centrifuging the medium and/or depth filtering the medium and/or filtering the medium through a 0.2 micron filter.

32. The aqueous liquid culture medium of claim 31 wherein the antibody binds specifically to IGF1R.

33. The aqueous liquid culture medium of claim 32 wherein the heavy immunoglobulin chain comprises the amino acid sequence set forth in a member selected from the group consisting of SEQ ID NOs: 1, 3,-6, 13, 21 and 26; or a mature fragment thereof or one or more CDRs therefrom;
and a light immunoglobulin chain comprises an amino acid sequence selected from the group consisting of SEQ ID NOs: 2, 7-9, 17 and 25, or a mature fragment thereof or one or more CDRs therefrom.

34. The aqueous liquid culture medium of claim 33 wherein the antibody or fragment is an antibody that is secreted from the host cell that comprises a light chain immunoglobulin comprising amino acids 20-128 of SEQ ID NO: 3, 4, 5, or 6; and a heavy chain immunoglobulin comprising amino acids 20-137 of SEQ ID NO: 8 or 9.

35. The aqueous liquid culture medium of claim 34 wherein the light chain immunoglobulin is linked to a kappa constant immunoglobulin chain and the heavy chain immunoglobulin is linked to a gamma-1 constant immunoglobulin chain.

36. A vessel comprising the aqueous liquid culture medium of claim 27.

37. The vessel of claim 36 which is a flask, a bioreactor, a tank bioreactor, a bag bioreactor or a disposable bioreactor.

38. The vessel of claim 37 which bioreactor is a stirred tank bioreactor, a bubble column bioreactor, an air lift bioreactor, a fluidized bed bioreactor or a packed bed bioreactor.


SEQUENCE LISTING (26 sequences)

SEQ ID NO: 1 - protein, 128 aa, Homo sapiens
MSPSQLIGFLLLWVPASRGEIVLTQVPDFQSVTPKEKVTITCRASQSIGSSLHWYQQKPDQSPKLLIKYASQSLSGVPSRFSGSGSGTDFTLTINSLEAEDAAAYYCHQSSRLPHTFGGGTKVEIKRT

SEQ ID NO: 2 - protein, 137 aa, Homo sapiens
MEFGLSWVFLVAILKGVQCEVQLVQSGGGLVHPGGSLRLSCAASGFTFSSFAMHWVRQAPGKGLEWISVIDTRGATYYADSVKGRFTISRDNAKNSLYLQMNSLRAEDMAVYYCARLGNFYYGMDVWGQGTTVTVSS

SEQ ID NO: 3 - protein, 128 aa, Homo sapiens
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDAAAYYCHQSSRLPHTFGQGTKVEIKRT

SEQ ID NO: 4 - protein, 128 aa, Homo sapiens
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDFAVYYCHQSSRLPHTFGQGTKVEIKRT

SEQ ID NO: 5 - protein, 128 aa, Homo sapiens
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDAAAYYCHQSSRLPHTFGQGTKVEIKRT

SEQ ID NO: 6 - protein, 128 aa, Homo sapiens
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRT

SEQ ID NO: 7 - protein, 137 aa, Homo sapiens
MEFGLSWVFLVAILKGVQCEVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 8 - protein, 137 aa, Homo sapiens
MEFGLSWVFLVAILKGVQCEVQLVQSGGGLVQPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 9 - protein, 144 aa, Homo sapiens
MELGLSWIFLLAILKGVQCEVQLVESGGGLVQPGRSLRLSCAASGFTFDDYAMHWVRQAPGKGLEWVSGISWNSGSKGYVDSVKGRFTISRDNAKNSLYLQMNSLRAEDTALYYCAKDIRIGVAASYYFGMDVWGHGTTVTVSS

SEQ ID NO: 10 - protein, 10 aa, Homo sapiens
GFTFDDYAMH

SEQ ID NO: 11 - protein, 17 aa, Homo sapiens
GISWNSGSKGYVDSVKG

SEQ ID NO: 12 - protein, 16 aa, Homo sapiens
DIRIGVAASYYFGMDV

SEQ ID NO: 13 - protein, 129 aa, Homo sapiens
MDMRVPAQLLGLLLLWLPGARCAIQLTQSPSSLSASVGDRVTITCRASQGISSVLAWYQQKPGKAPKLLIYDASSLESGVPSRFSGSGSGTDFTLTISSLQPEDFATYYCQQFNSYPYTFGQGTKLEIK

SEQ ID NO: 14 - protein, 11 aa, Homo sapiens
RASQGISSVLA

SEQ ID NO: 15 - protein, 7 aa, Homo sapiens
DASSLES

SEQ ID NO: 16 - protein, 9 aa, Homo sapiens
QQFNSYPYT

SEQ ID NO: 17 - protein, 146 aa, Homo sapiens
MDWTWRILFLVAAATGAHSQVQLVQSGAEVKKPGASVKVSCKASGYTFTSYVMHWVRQAPGQRLEWMGWINAGNGNTKYSQKFQGRVTITRDTSASTVYMELSSLRSEDTAVYYCARGGMPVAGPGYFYYYGMDVWGQGTTVTVSS

SEQ ID NO: 18 - protein, 10 aa, Homo sapiens
GYTFTSYVMH

SEQ ID NO: 19 - protein, 17 aa, Homo sapiens
WINAGNGNTKYSQKFQG

SEQ ID NO: 20 - protein, 18 aa, Homo sapiens
GGMPVAGPGYFYYYGMDV

SEQ ID NO: 21 - protein, 130 aa, Homo sapiens
METPAQLLFLLLLWLPDTTGEIVLTQSPGTLSLSPGERATLSCRASQSVSRSYLAWYQQKPGQAPRLLIYGASSRATGIPDRFSGSGSGTDFTLTISRLEPEDFAVYCCQQYGSSPWTFGQGTKVEIKRT

SEQ ID NO: 22 - protein, 12 aa, Homo sapiens
RASQSVSRSYLA

SEQ ID NO: 23 - protein, 7 aa, Homo sapiens
GASSRAT

SEQ ID NO: 24 - protein, 9 aa, Homo sapiens
QQYGSSPWT

SEQ ID NO: 25 - protein, 125 aa, Homo sapiens [sequence omitted]

SEQ ID NO: 26 - protein, 108 aa, Homo sapiens
DIQMTQFPSSLSASVGDRVTITCRASQGIRNDLGWYQQKPGKAPKRLIYAASRLHRGVPSRFSGSGSGTEFTLTISSLQPEDFATYYCLQHNSYPCSFGQGTKLEIKR